(19) United States
(12) Patent Application Publication
(10) Pub. No.: US 2023/0230636 A1
(43) Pub. Date: Jul. 20, 2023

(54) NANOPORE UNZIPPING-SEQUENCING FOR DNA DATA STORAGE

(71) Applicant: The Curator's of the University of Missouri, Columbia, MO (US)

(72) Inventors: Li-Qun GU, Columbia, MO (US); Kai TIAN, Columbia, MO (US)

(21) Appl. No.: 17/998,269

(22) PCT Filed: May 14, 2021
(86) PCT No.: PCT/US21/32538

§ 371 Date: Nov. 9, 2022

Related U.S. Application Data
(60) Provisional application No. 63/025,402, filed on May 15, 2020.

(51) Int. Cl. G11C 13/00; G01N 33/487

(52) U.S. Cl. CPC G11C 13/0019; G01N 33/48721; B82Y 15/00

(57) ABSTRACT

The present disclosure relates to methods of writing data in nucleic acid chains and methods of reading data written in nucleic acid chains. The present disclosure also relates to a kit for writing and reading data in nucleic acid chains.

### CROSS-REFERENCE TO RELATED APPLICATION

This application claims priority from U.S. Provisional Patent Application No. 63/025,402, filed May 15, 2020, which is incorporated herein by reference in its entirety.

### STATEMENT REGARDING FEDERALLY SPONSORED RESEARCH OR DEVELOPMENT

This invention was made with Government support under NIH BM114204 and NIH HG009338 awarded by the National Institute of Health. The Government has certain rights in the invention.

### STATEMENT IN SUPPORT FOR FILING A SEQUENCE LISTING

A paper copy of the Sequence Listing and a computer readable form of the Sequence Listing containing the file named “20UMC035.txt”, which is 20,985 bytes in size (as measured in MICROSOFT WINDOWS® EXPLORER), are provided herein and are herein incorporated by reference. This Sequence Listing consists of SEQ ID NOs:1-57.

### BACKGROUND

The present disclosure relates generally to methods of writing data in nucleic acid chains, and methods of reading data written in nucleic acid chains. The present disclosure also relates to a kit for writing and reading data in nucleic acid chains.

In the era of explosive digital data growth, DNA is being explored as a next-generation molecular storage media. One can encode data in vitro by employing the four natural nucleotides (A, T, G, and C), synthesizing data in a DNA strand, and retrieving data from the DNA by sequencing. Due to molecular manipulation at the atomic level, DNA data storage achieves extremely high data density up to 10¹⁸ bytes per cubic millimeter (1 uL), 6 orders of magnitude denser than the densest media available today. DNA material is extremely stable both in liquid or dry paper at a relatively high temperature, offering high durability (high retention) over many current media materials. Data in DNA can easily generate billions of copies via a simple PCR reaction while maintaining low energy. DNA data retrieval greatly benefits from the revolution of sequencing technology, including Illumina Next Generation Sequencing (NGS) and Nanopore 3^{rd} generation Sequencing, which can rapidly sequence human and other genomes at ever-decreasing prices.

However, current strategies for DNA data storage also confront challenges. Storage of any dataset needs template-free synthesis of specific long DNA molecules either by chemical or enzymatic methods, which remains highly expensive, time-consuming, labor intensive, and error-prone. Synthesized DNA cannot be re-used to store other datasets, further increasing the storage cost. For data retrieving, synthesis-based Illumina sequencing has to cut long data DNA molecules into short fragments (<300 bases) to keep a low error rate (<0.1%), and requires complicated post-sequencing bioinformatic analysis to assemble fragmented data.

For these reasons, efforts have been made to write data into a universal DNA sequence including native DNA. Recently, Chen et al constructed a string of protein (streptavidin)-labeled and unlabeled DNA nanostructures on template DNA to encode binary data, and used a nanopore-terminated glass nanopipette to identify the protein-labeled DNA nanostructure signal that is distinct from unlabeled DNA nanostructures and background signal, thereby decoding binary data. This method eliminates long DNA synthesis in data writing, but the storage of each bit still needs protein labeling, which increases the cost. Also, under current glass nanopore resolution, only binary data represented by the labeled and unlabeled states for each bit can be read, and neighboring protein labels have to be separated enough to distinguish from each other which lowers data density. Tabatabaei et al reported transformation of binary data into a series of positions along with native DNA and utilized the nickase PfAgo with synthetic phosphorylated guide DNA to form nicks at these data positions for data writing. Sequencing all the fragmented DNA by NGS allowed determining the nick reaction occurrence at each position, thereby retrieving the data. This data writing strategy also eliminates long DNA synthesis but requires parallel enzyme reactions for each bit which may cause non-specific nicking. Additionally, enzymatic treatment is considerably costly and time-consuming. Experimentally, data reading still relies on sequencing-by-synthesis approaches (NGS) followed by complicated sequence alignment.

These shortcomings highlight the need for new methods for reading and storing data.

### SUMMARY

The present disclosure relates generally to methods of writing data in nucleic acid chains, and methods of reading data stored in nucleic acid chains. The present disclosure also relates to a kit for writing and reading data in nucleic acid chains.

In one aspect, the present disclosure is directed a method for reading stored data, the method comprising:
directing a portion of a nucleic acid chain into a nanopore, wherein the chain represents stored data and comprises codons, addresses, and blockers, wherein the codons each comprise one or more unpaired nucleotides and wherein the addresses and the blockers each comprise segments of nucleotides, wherein the blockers are each configured to be bound with a corresponding one of the addresses, and wherein the nanopore comprises a constricting region;
applying an electric potential across the nanopore to move the chain through the nanopore, wherein the chain moves through the nanopore until a first codon of the chain enters the constricting region and a first blocker of the chain and a corresponding first address of the chain bound thereto encounters the constricting region, and wherein the first blocker and its corresponding first address encountering the constricting region stops the movement of the chain;
measuring a first current in the nanopore when the first codon is in the constricting region;
dissociating the first blocker from its corresponding first address bound thereto to resume the movement of the chain through the nanopore;
repeating the measuring step and the dissociating step with additional codons to measure additional currents; and
translating the measured currents into an output signal representative of the stored data.

In another aspect, the present disclosure is directed to a method for writing data, the method comprising:
providing a nucleic acid chain, wherein the chain comprises a plurality of coding windows and addresses, wherein the coding windows each comprise three or more unpaired nucleotides and the addresses each comprise three or more unpaired nucleotides;
binding the addresses to blockers, wherein the blockers each comprise an address match and an encoder, the address match complements the nucleotides of the addresses, and the encoder complements nucleotides of the coding windows adjacent to the addresses; and
defining codons based on the address match and the encoder to encode data into at least a portion of the chain, wherein the codons each comprise two or more nucleotides of the coding window preceding the nucleotides bound with the blocker.

In another aspect, the present disclosure is directed to another method for writing data, the method comprising:
providing a nucleic acid chain, wherein the chain comprises a plurality of coding windows and addresses, wherein the coding windows each comprise three or more unpaired nucleotides and the addresses each comprise three or more unpaired nucleotides;
using a microfluidic device or robot to collect blockers from a large blocker pool in a programmable and parallel manner, wherein the blockers each comprise an address match and an encoder, the address match complements the nucleotides of the addresses, and the encoder complements nucleotides of the coding windows adjacent to the addresses;
binding the addresses to blockers; and
defining codons based on the address match and the encoder to encode data into at least a portion of the chain, wherein the codons each comprise two or more nucleotides of the coding window preceding the nucleotides bound with the blocker.

In another aspect, the present disclosure is directed to a method for encoding data and reading the encoded data, the method comprising
providing a nucleic acid chain, wherein the chain comprises a plurality of coding windows and addresses, wherein the coding windows each comprise three or more unpaired nucleotides and the addresses each comprise three or more unpaired nucleotides;
binding the addresses to blockers, wherein the blockers each comprise an address match and an encoder, the address match complements the nucleotides of the addresses, and the encoder complements nucleotides of the coding windows adjacent to the addresses;
defining codons based on the address match and the encoder to encode data into at least a portion of the chain, wherein the codons each comprise two or more nucleotides of the coding window preceding the nucleotides bound with the blocker; and
reading the encoded data.

In another aspect, the present disclosure is directed to a kit for writing and reading data, the kit comprising:
a universal nucleic acid chain comprising coding windows and addresses, wherein the coding windows comprise three or more unpaired nucleotides and the addresses comprise three or more unpaired nucleotides;
blockers comprising an address match and an encoder, wherein the address match comprises nucleotides complementing the nucleotides of the addresses and the encoder comprises nucleotides complementing nucleotides of the coding windows adjacent to the addresses, and the blockers are configured such that they may bind to the addresses and thereby define codons in the coding windows such that the codons comprise two or more nucleotides of the coding window preceding the nucleotides bound with the blocker;
a microfluidic device comprising:
   an inlet for receiving a flow comprising the chain, and
   a plurality of nanopores comprising a constricting region configured such that applying an electric potential across the nanopore causes the chain to move through the nanopore until a first codon enters the constricting region and a first blocker encounters the constricting region and temporarily stops the movement of the chain; and
a measuring device for measuring the current through the nanopore.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 depicts a coupled frameshift encoding/NP Unzip-Seq decoding strategy for data storage/retrieval from a universal DNA according to an embodiment.

FIG. 2 depicts a nanopore unzipping-sequencing (NP Unzip-Seq) platform according to an embodiment.

FIG. 3A depicts structures of eight probing DNAs from D9TOC to D2T7C and schematics of their configurations in the nanopore according to an embodiment.

FIG. 3B depicts representative nanopore current traces showing blockages and enlarged blockades for all of the eight probing DNAs of FIG. 3A.

FIG. 3C depicts histograms of signature blocking levels as a function of the number of T→C substitutions for all the probing DNAs of FIG. 3A.

FIG. 3D depicts values of signature blocking levels as a function of the number of T→C substitutions for all the probing DNAs of FIG. 3A.

FIG. 4A depicts conformations and nanopore signatures for three steps in codon reading according to an embodiment.

FIG. 4B depicts sequentially reading 3-bit binary codons according to an embodiment.

FIG. 4C depicts reading a long codon template by NP Unzip-Seq that encodes the 8-bit binary ASCII code of the letter ‘M’ according to an embodiment.

FIG. 5A depicts a schematic for frameshift encoding according

to an embodiment.

FIG. 5B depicts schematics for writing 3-bit binary numbers (000, 001, 010, and 011) in a universal DNA by using different panels of staples and nanopore current signatures for reading these numbers according to an embodiment.

FIG. 6A depicts sequences of four data unit segments randomly truncated from the M13 DNA to form a universal template according to an embodiment.

FIG. 6B depicts sequences of the template of FIG. 6A in complex with staples showing formation of multiple codons in each coding window by staple-facilitated frameshifting base by base and nanopore current signature showing sequential readout of the four codons by NP Unzip-Seq.

FIG. 6C depicts blocking levels for the six codons of FIG. 6B that were formed in each coding window by frameshifting for the four coding windows.

FIG. 6D depicts selection of four nanopore-discriminable codons among the six codons of FIG. 6B in each coding window to form various quartet codon panels for quaternary data encoding.

FIG. 7A depicts a schematic of automatic identification of coding windows in a native DNA sequence according to an embodiment.

FIG. 7B depicts a schematic of encoding data into M13 genomic DNA by the coding windows identified in FIG. 7A.

FIGS. 8A-8C depict 78 highlighted coding window sequences in M13 DNA for quaternary encoding according to an embodiment.

FIGS. 8D-8F depict 201 highlighted coding window sequences in M13 DNA for octal encoding according to an embodiment.

FIG. 9A depicts a schematic and nanopore current signature for reading the eight codons from the D-Octal by NP Unzip-Seq according to an embodiment.

FIG. 9B depicts current histograms of the eight codons of FIG. 9A.

FIG. 9C depicts a diagram showing an octonary 8-pixel grey level image and grey-scale color image encoded by D-Octal according to an embodiment.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

The approach of the present disclosure is to store data in DNA by using blockers to write a codon sequence on a nucleic acid sequence. The codon sequence is read codon-by-codon on a nanopore unzipping-sequencing (NP Unzip-Seq) platform. This coupled encoding and NP Unzip-Seq method surprisingly writes, reads, and rewrites not only binary but also multinary data in a fast, enzyme-free, and label-free manner without the need for long DNA synthesis.

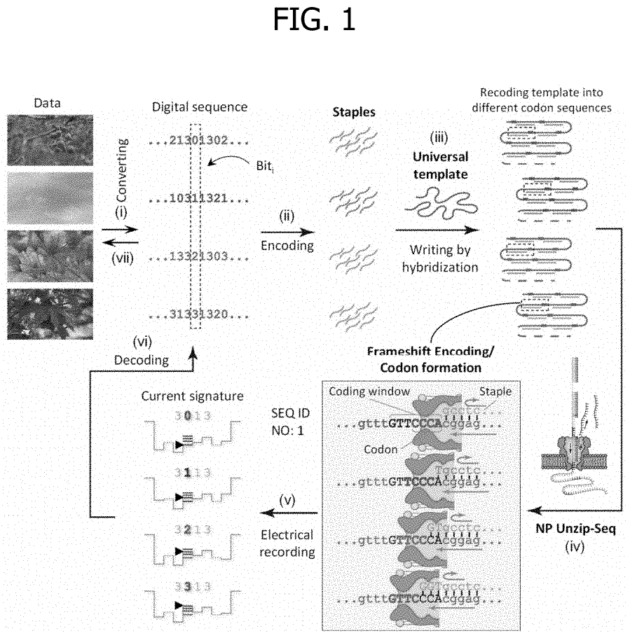

The strategy is summarized by FIG. 1. (i) A target dataset is converted to a digital sequence. (ii) A set of blockers (i.e., oligo staples) that encode the digital sequence is collected from a synthetic blocker library. (iii) The blockers are hybridized with a nucleic acid chain to define a codon sequence that stores the data. (iv) To retrieve the data, the blocker-bound chain is read codon-by-codon by NP Unzip-Seq, which (v) records an electrical signature for each codon. (vii) The codon sequence is decoded into digital sequence, which (vii) is converted back to the data.

### Reading Data

In this data storage/retrieval strategy, data is encoded into a nucleic acid chain by defining sequences of codons, and a nanopore is used to decode the data codon-by-codon.

An aspect of the present disclosure is directed to a method for reading stored data, the method comprising:
directing a portion of a nucleic acid chain into a nanopore, wherein the chain represents stored data and comprises codons, addresses, and blockers, wherein the codons each comprise one or more unpaired nucleotides and wherein the addresses and the blockers each comprise segments of nucleotides, wherein the blockers are each configured to be bound with a corresponding one of the addresses, and wherein the nanopore comprises a constricting region;
applying an electric potential across the nanopore to move the chain through the nanopore, wherein the chain moves through the nanopore until a first codon of the chain enters the constricting region and a first blocker of the chain and a corresponding first address of the chain bound thereto encounters the constricting region, and wherein the first blocker and its corresponding first address encountering the constricting region stops the movement of the chain;
measuring a first current in the nanopore when the first codon is in the constricting region;
dissociating the first blocker from its corresponding first address bound thereto to resume the movement of the chain through the nanopore;
repeating the measuring step and the dissociating step with additional codons to measure additional currents; and
translating the measured currents into an output signal representative of the stored data.

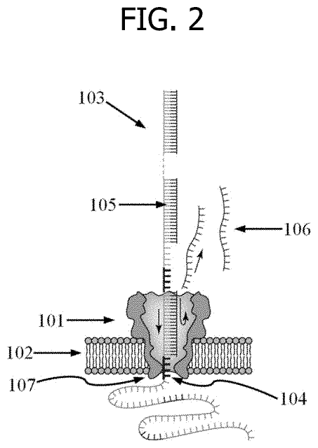

The nanopore unzipping-sequencing (NP Unzip-Seq) platform is illustrated in FIG. 2. The nanopore (101) is a small opening in a biological or solid-state membrane (102) surrounded by electrolyte solution. The membrane (102) splits the solution into two chambers. An electric potential is applied across the membrane (102) inducing an electric field that drives a nucleic acid chain (103) into motion through the nanopore (101). Inside the nanopore (101), the nucleic acid molecule (103) occupies a volume that partially restricts the flow of ions, resulting in a current drop. Based on various factors such as geometry, size and chemical composition, the change in magnitude of the current will vary. Different codons (104) can be sensed and identified based on this modulation in current.

Still referring to FIG. 2, each codon (104) is associated with an address (105) that is a short segment of the nucleic acid chain (101) downstream of the codon (104). The address can bind a blocker (106) (i.e. an oligonucleotide) to form a double stranded segment. Single stranded segments of the nucleic acid chain (e.g. the codon (104)) easily pass through a narrow, constricting region (107) of the nanopore (101). Double stranded segments (e.g the address (105) with a bound blocker (106)) cannot easily pass through the constricting region (107), but can be unbound due to the applied electric potential to release a single stranded segment that can pass through. Accordingly, driven by the electric potential, each codon (104) is pulled into the nanopore (101), is immobilized temporarily in the constricting region (107) by the blocker (106) while a characteristic current is read, and finally is moved through the nanopore (101) once the blocker (106) is unbound.

In some embodiments, the nanopore is a protein nanopore. Suitable protein nanopores include but are not limited to MspA nanopores, CsgG nanopores, and a-hemolysin nanopores. In some embodiments, the MspA nanopore is an MspA mutant nanopore, for example MspA-M2. In some embodiments, the nanopore is a solid nanopore, a synthetic nanopore, a hybrid nanopore, or other nucleotide-sensitive nanochannels. In some embodiments, the hybrid nanopore incorporates a protein nanopore in a solid nanopore.

In some embodiments, dissociating the first blocker from its address comprises performing enzyme-free vectorial unzipping. In some embodiments, the electric potential is from about 50 mV to about 200 mV, about 50 mV to about 150 mV, about 50 mV to about 120 mV, about 100 mV to about 200 mV, about 100 mV to about 150 mV, or about 100 mV to about 120 mV. In some embodiments, the electric potential is about 100 mV, about 120 mV, or about 150 mV. In some embodiments, the electric potential is up to several volts.

As used herein, codons refer to sequences of the nucleic acid chain encoding data. The codons each include 1 or more unpaired nucleotides. In some embodiments, the codons each include 2 or more unpaired nucleotides. In some embodiments, the codons each include 1 to 5, 1 to 4, 1 to 3, 2 to 5, 2 to 4, 2 to 3, 3 to 5, or 3 to 4 unpaired nucleotides. In some embodiments, the codons each include 1, 2, 3, 4, 5 nucleotides, or combinations thereof. In some embodiments, the codons each include up to 10 nucleotides. In some embodiments, the nanopore conductance was found to be sensitive to the first 4 unpaired nucleotides of the nucleic acid chain upstream of the blocker. Therefore, particularly suitable codons each include 4 unpaired nucleotides.

The conductance of the nanoparticle pore is highly sensitive to the identity and sequence of nucleotides in the codon. A change to a single nucleotide in the codon causes a characteristic change in the nanopore conductance. The identity of the codon can be read out from the nanopore current signature (see FIG. 1, step (v), Electrical Recording). Accordingly, in some embodiments, the first current of the first codon has a characteristic current pattern associated with the sequence of the nucleotides of the first codon. In some embodiments, the characteristic current pattern includes one or more of a characteristic amplitude, a characteristic duration, characteristic noise, and a characteristic subconductance. To ensure selected codons are discriminable from each other, in some embodiments, a nanopore current difference between any two codons is larger than a cut-off value ΔI_{shift}, wherein ΔI_{shift} is a measure of discriminability at the instrument resolution. A large ΔI_{shift} increases codon discrimination accuracy, but results in fewer qualified codons.

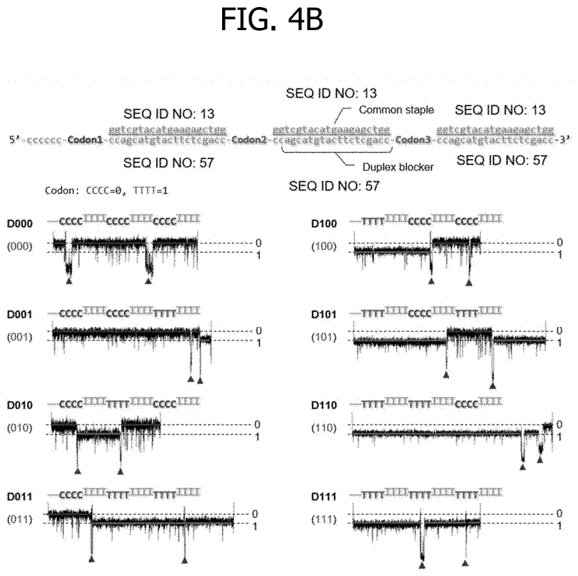

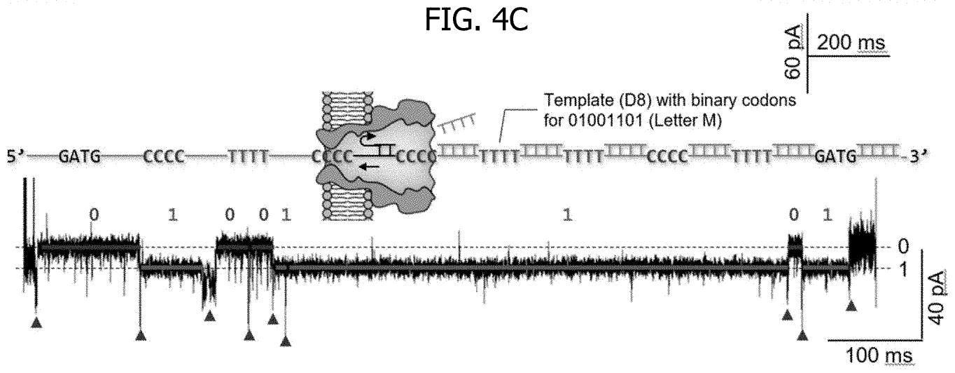

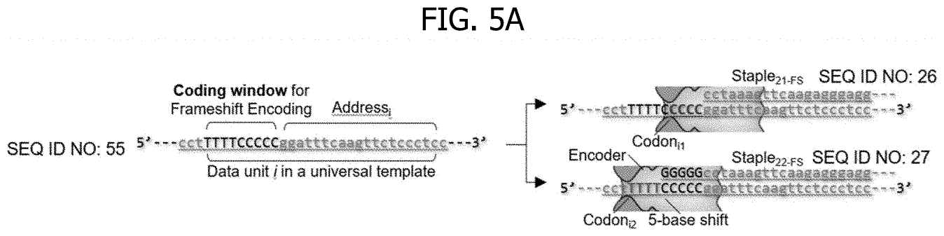

The NP Unzip-Seq platform can discriminate the starts/stops of codons, including between codon repeats that are otherwise difficult to distinguish by current sequencing approaches (for example, nanopore sequencing methods struggle to identify repeating sequences because each sequence produces the same current read). In the present methods, distinctively lower conductance was briefly observed between codons (i.e., inter-codon markers as shown in FIG. 4A.) Without being bound by particular theory, it is believed that the inter-codon markers are generated by (1) the blocker, which remains trapped in the nanopore after fully or partially unbinding from the address and co-occupying the nanopore lumen with the next duplex; or by (2) the unbinding address, which is grasped by the highly positively charged domain of the nanopore during transient unzipping, causing the unbound part of the address to ‘curl’ when pushed by the voltage. Accordingly, in some embodiments, the method further includes measuring a second current after the first blocker dissociates from its address and before a second codon enters the constricting region. In some embodiments, the second current is used to demarcate the first current and a third current associated with the second codon. In some embodiments, the method further includes identifying one or more repeating patterns in the measured currents to identify one or more repeating codons in the chain. While not required, an embodiment could further include in the method a helicase or other motor enzymes to control the passing of the DNA codon by codon.

FIG. 4A depicts NP Unzip-Seq with intercodon markers: (i) a codon immobilized in the sequence-reading zone of the pore and an associated current read, (ii) an inter-codon marker is produced when the blocker is released, and (iii) the next codon in the chain pulled into the nanopore and an associated current read.

As used herein, blocker refers to an oligonucleotide that binds the nucleic acid chain to define codons. Preferably, the blocker binds the nucleic acid chain at or about an address on the nucleic acid chain. Suitable blockers each comprise about 5 to about 30 nucleotides. In some embodiments, the blockers each comprise about 5 to about 30, about 5 to about 25, about 5 to about 20, about 10 to about 30, about 10 to about 25, about 10 to about 20, about 15 to about 30, about 15 to about 25, or about 15 to about 20 nucleotides. In some embodiments, the blockers each comprise 10, 11, 12, 13, 14, 15, 16, 17, 18, 19, 20, 21, 22, 23, 24, 25, 26, 27, 28, 29, 30 nucleotides or combinations thereof.

As used herein, address refers to a short segment of the nucleic acid chain that binds a blocker to define a codon upstream of the address. Suitable addresses each comprise about 5 to about 30 nucleotides. In some embodiments, the addresses each comprise about 5 to about 30, about 5 to about 25, about 5 to about 20, about 10 to about 30, about 10 to about 25, about 10 to about 20, about 15 to about 30, about 15 to about 25, or about 15 to about 20 nucleotides. In some embodiments, the addresses each comprise 10, 11, 12, 13, 14, 15, 16, 17, 18, 19, 20, 21, 22, 23, 24, 25, 26, 27, 28, 29, 30 nucleotides or combinations thereof. In some embodiments, the addresses of the nucleic acid chain are all identical. In other embodiments, the nucleic acid chain has multiple the nucleotides of the addresses have an address sequence and the chain comprises two or more address sequences.

Nucleotides compatible with aspects of the invention may be any nucleotides, derivatives, or nucleotide-like compounds as are known in the art. In some embodiments, the nucleotides are natural nucleotides (A, T, G, C). In some embodiments, the nucleotides are artificial nucleotides such as LNA, BNA, and PNA. In some embodiments, the nucleotides are modified nucleotides such as methylated nucleotides. In some aspects, the nucleotides are DNA nucleotides. In some embodiments, the nucleotides are RNA nucleotides.

### Writing Data

In this data writing/reading strategy, data is written into a nucleic acid chain by defining sequences of codons.

An aspect of the present disclosure is directed to a method for writing data, the method comprising:
providing a nucleic acid chain, wherein the chain comprises a plurality of coding windows and addresses, wherein the coding windows each comprise three or more unpaired nucleotides and the addresses each comprise three or more unpaired nucleotides;
binding the addresses to blockers, wherein the blockers each comprise an address match and an encoder, the address match complements the nucleotides of the addresses, and the encoder complements nucleotides of the coding windows adjacent to the addresses; and
defining codons based on the address match and the encoder to encode data into at least a portion of the chain, wherein the codons each comprise two or more nucleotides of the coding window preceding the nucleotides bound with the blocker.

As used herein, coding window refers to a short segment of the nucleic acid chain upstream of the address. The blocker defines a codon in the coding window when the blocker binds the address. In some embodiments, the coding windows each comprise about 1 to about 20, about 1 to about 10, about 1 to about 5, about 2 to about 20, about 2 to about 10, about 2 to about 5, about 3 to about 20, about 3 to about 10, about 3 to about 5, about 4 to about 20, about 4 to about 10, about 5 to about 20, or about 5 to about 10 nucleotides.

Frameshift encoding. Since the blocker binding to the nucleic acid chain controls the codon formation, extending or shortening the blocker length by n nucleotides relative to the address length enables shifting the codon frame backward or forward by the same number of bases. This frameshift encoding strategy enables defining different in the coding window without changing the address or coding window sequence.

Accordingly, in some embodiments, the blocker comprises an address match and an encoder. The address match complements the nucleotides of the addresses. The encoder complements nucleotides of the coding windows adjacent to the addresses. Encoders of different lengths are able to shift the codon frame by different numbers of bases, generating multiple codons within the coding window at each address.

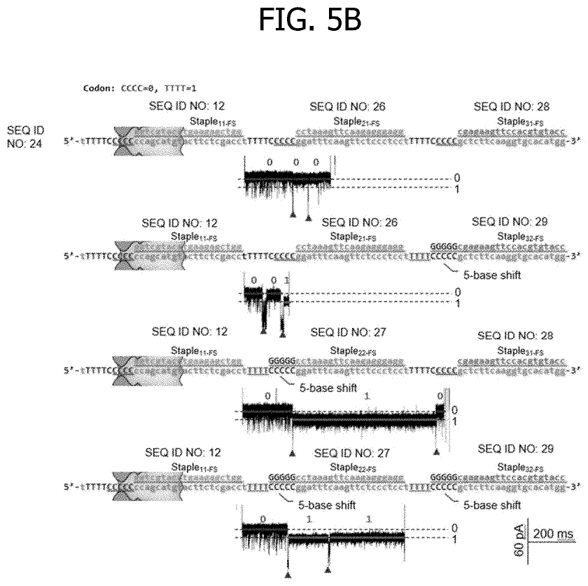

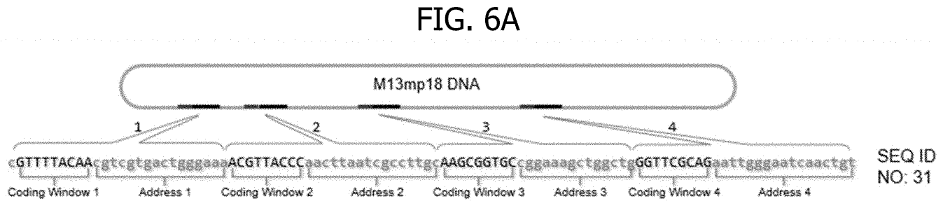

The frameshift encoding strategy is demonstrated in FIG. 5A. (Left) Shows a nucleic acid chain including a coding window and an address. (Right) Shows showing binary encoding by frameshift. Stapleᵢ₁ has an encoder with 0 nucleotides (null). Stapleᵢ₂ has an encoder with 5 nucleotides (GGGGG) at the 3′ end. Upon binding to the address, the encoders ‘silence’ 0 and 5 nucleotides in the coding window, shifting the codon frame by 0 and 5 bases to form codons CCCC and TTTT, respectively.

In some embodiments, the method further includes defining codons based on the address match and the encoder comprises shifting the coding window along the chain by a predetermined number of bits. In some embodiments, the encoder comprises 0 or more nucleotides and a size of the encoder determines the number of bits by which the coding window is shifted. In some embodiments, the encoders each comprise about 0 to about 10, about 0 to about 5, about 1 to about 10, or about 1 to about 5 nucleotides. In some embodiments, the encoders each comprise 0, 1, 2, 3, 4, 5, 6, 7, 8, 9, 10 nucleotides and combinations thereof.

By writing data in multinary format, data storage density is greatly increased. Frameshift encoding enables encoding data in multinary format on the nucleic acid chain. To encode an n-nary dataset (n is 2, 4, 8 and 16 for binary, quaternary, octal and hexadecimal data, respectively) each coding window forms n different codons by frameshift. In some embodiments, the output is multinary. In some embodiments, the output is quaternary, octal, or hexadecimal. For example, to encode data in quaternary format, in some embodiments, a set of four blockers each have an identical address match but each have a unique encoder including 0, 1, 2 or 3 nucleotides. Upon binding to the same address sequence, these staples shift the codon frame by 0, 1, 2 and 3 bases respectively to form four different codons with bit values of 0, 1, 2, and 3. In some embodiments, the output is binary.

Another aspect of the present disclosure is directed to a method for encoding data and reading the encoded data, the method comprising
providing a nucleic acid chain, wherein the chain comprises a plurality of coding windows and addresses, wherein the coding windows each comprise three or more unpaired nucleotides and the addresses each comprise three or more unpaired nucleotides;
binding the addresses to blockers, wherein the blockers each comprise an address match and an encoder, the address match complements the nucleotides of the addresses, and the encoder complements nucleotides of the coding windows adjacent to the addresses;
defining codons based on the address match and the encoder to encode data into at least a portion of the chain, wherein the codons each comprise two or more nucleotides of the coding window preceding the nucleotides bound with the blocker; and
reading the encoded data using the methods disclosed herein.

### Nucleic Acid Chain

Universal template. In some embodiments, the nucleic acid chain is a universal template. The universal template is a 1D array of data units. Each data unit consists of a coding window followed by an address. In some embodiments, the nucleotides of the coding windows have a coding window sequence and the coding window sequence is the same for all the coding windows.

Frameshift encoding allows different codons to be defined (and hence different data to be written) on the universal template without changing its sequence. Thus, frameshift encoding enables a universal template to encode various datasets by shifting the codon reading frame without the need for DNA synthesis.

In some embodiments, the frameshift encoding strategy is applied in writing and reading multinary data in a native DNA that functions as a hard drive. In such embodiments, the universal template is native DNA or modified native DNA, wherein long single-stranded templates are extracted from the double-stranded DNA. For example, in some embodiments the nucleic acid chain is DNA from organisms (e.g., naturally-sourced DNA derived from recombining several fragments of viral DNA). Particularly suitable organisms are bacteria and viruses, due to the advantages of large-scale native DNA proliferation and extraction at low cost and high efficiency.

To use a native DNA as the universal template, in some embodiments the methods include identifying qualified coding window sequences in the native DNA sequences. In some embodiments, the criteria for identifying qualified coding windows sequences include capacity, step, and conductance difference between two codons formed in a coding window.

Capacity. To encode an n-nary dataset (n is 2, 4, 8 and 16 for binary, quaternary, octal and hexadecimal data), each coding window should be able to form n different (nanopore-discriminable) codons by frameshift; i.e. the capacity is n.

Step. The coding window sequences are also dependent on the number of bases for each shift, i.e. step. In some embodiments, step is 1. A search with multiple Step values may result in more qualified candidate coding window sequences. If the frameshift step is set to 1-nt, a coding window needs to contain at least n+3 nucleotides.

Conductance. Finally, the nanopore current difference produced between any two codons should be larger than a cut-off value ΔI_{shift}, a measure of discriminability at the instrument resolution. A large ΔI_{shift} increases codon discrimination accuracy, but also results in fewer qualified candidate coding window sequences. After coding windows are identified, blockers are synthesized with encoders for encoding multinary data in the coding windows by frameshift encoding.

Barcodes. In another aspect, the nucleic acid chain is used for one or more of labelling and identification of a biomarker. In some embodiments, the nucleic acid chain is a barcode. The barcode labels biomarkers such that NP-Unzip Seq recognizes the barcode to identify the biomarker. In some embodiments, biomarker is a nucleic acids fragment (e.g. a native genome DNA or RNA fragment containing driver mutations), a pathogenic DNA or RNA fragment (e.g. from a bacterium and virus), a panel of microRNAs, long non-coding RNAs, or other nucleic acids sequences of interest. In some embodiments, the biomarker is a protein, a peptide, or a small molecule such as metabolites and metal ions. In some embodiments, the biomarker labels a probe or a receptor that labels the biomarker. For example, in some embodiments, the biomarker is a nucleic acid, the probe is a complementary strand that binds the nucleic acid biomarker, and the barcode labels the probe.

Native nucleic acid chains. In another aspect, the nucleic acid chain comprises a native nucleic acid chain. In some embodiments, the nucleic acid chain is a native nucleic acid chain. Suitable native nucleic acids chains are DNA and RNA. In some embodiments, the methods include converting double stranded DNA into single stranded DNA by any known means (e.g. heating). In some embodiments, the methods include identifying one or more of genetic alterations and epigenetic alterations in the nucleic acid chain. Genetic alterations include single nucleotide polymorphism, insertions, deletions, frameshift mutations, duplications, and repeat expansions. Epigenetic alterations include oxidative stress and methylation. In some embodiments, the methods are used to study the functions of enzymes that produce these alterations and their relations to diseases.

For example, DNA methylation occurs in clusters (CpG islands) in promotor regions. RNA methylation also has consensus sequences. Some embodiments are directed to identifying methylation sites in the nucleic acid chain. Such methods include designing a set of blockers to bind the nucleic acid sequence near (but not over) a potential methylation site. The blockers define codons, and some of the codons include methylation sites. Nanopore Unzip-Seq sequentially reads these codons while sequentially unzipping these blockers one by one, such that the status of each codon (i.e. with or without methylation) is identified. In some embodiments, the methods include performing statistical analysis of the result of many nucleic acids chains. In some embodiments, the methods include quantifying the overall methylation percentage distribution. In some embodiments, the methods include quantifying the locus-specific methylation occurrence probability.

RNA. In another aspect, the nucleic acid sequence is an RNA sequence. RNA includes double-stranded motifs that are interconnected via canonical and non-canonical interactions. In some embodiments, the methods include reading a sequence before a double-stranded motif. In some embodiments, the methods include sequentially unzipping all the double-stranded motifs along the RNA chain to read the sequence before each motif. In some embodiments, the methods include mapping the locations of all the motifs formed in the RNA. In some embodiments, the nucleic acid sequence is an RNA sequence and the method includes identifying secondary and tertiary structures in the RNA sequence.

### Kits

Another aspect of the present disclosure is directed to a kit for writing and reading data, the kit comprising:
a universal nucleic acid chain comprising coding windows and addresses, wherein the coding windows comprise three or more unpaired nucleotides and the addresses comprise three or more unpaired nucleotides;
blockers comprising an address match and an encoder, wherein the address match comprises nucleotides complementing the nucleotides of the addresses and the encoder comprises nucleotides complementing nucleotides of the coding windows adjacent to the addresses, and the blockers are configured such that they may bind to the addresses and thereby define codons in the coding windows such that the codons comprise two or more nucleotides of the coding window preceding the nucleotides bound with the blocker;
a microfluidic device comprising:
   an inlet for receiving a flow comprising the chain, and
   a plurality of nanopores comprising a constricting region configured such that applying an electric potential across the nanopore causes the chain to move through the nanopore until a first codon enters the constricting region and a first blocker encounters the constricting region and temporarily stops the movement of the chain; and
a measuring device for measuring the current through the nanopore.

### EXAMPLES

Examples 1-3 are directed to methods used in the rest of the examples. Examples 4-10 are directed to reading data encoded in a nucleic acid chain. Example 11 is directed to writing data on a nucleic acid chain via frameshift encoding. Examples 12-15 are directed to frameshift encoding and decoding multinary data in native DNA sequences. Example 16 is directed to the advantages of this invention.

Examples 1-3 are directed to methods used in the rest of the examples.

### Example 1

This example presents a method of preparing MspA protein that is used in other examples.

The mutated MspA porin (D90N/D91N/D93N/D118R/ D134R/E139K) was prepared by previously reported methods (Yan et al., 2019; Wang et al., 2018; Heinz et al., 2003; Butler et al., 2008). The gene of mutated MspA with poly-histidine tag (H6) on the C-terminal was synthesized and cloned into a pET-30a(+) plasmid by Genscript. Competent cells of E. coli BL21 (DE3) were transformed with the plasmid by heat shock and were plated on LB agar supplemented with kanamycin and incubated at 37° C. overnight. A single colony was picked and grew in LB medium with kanamycin. Until OD600=0.7, the cells were induced with 1 mM isopropyl β-D-thiogalactoside (IPTG) and shaken overnight at 16° C. They were harvested by centrifugation at 4000 rpm 30 min at 4° C. The pellets were lysed in the lysis buffer (100 mM Na₂HPO₄/NaH₂PO₄, 0.1 mM EDTA, 150 mM NaCl, 0.5% (w/v) Genapol pH 6.5) at 60° C. for 10 min. The centrifuge tubes were kept on ice for 10 min and centrifuged at 10,000 rpm for 30 min at 4° C. After syringe filtration with a 0.22 μm filter, the supernatant was transferred to a nickel affinity column (HisTrap™ HP). The column was washed by washing buffer (0.5 M NaCl, 20 mM HEPES, 5 mM imidazole, 0.5% (w/v) Genapol X-80, pH=8.0). The MspA proteins were eluted by eluting buffer (500 mM imidazole, 0.5 M NaCl, 20 mM HEPES, 0.5% (w/v) Genapol X-80, pH=8.0). The solution with a gradient concentration of imidazole was collected in different EP tubes. The assembly of MspA in each tube was characterized by 12% SDS-PAGE.

### Example 2

This example presents a method of DNA hybridization that is used in other examples.

DNA fragments were synthesized by Integrated DNA Technologies, Inc. The DNAs were resolved in deionized water to 200 μM and diluted by the same volume of salt solution (200 mM KCl, 20 mM Tris-Cl, pH 8.0) to 100 μM. Besides the experiments of discrimination of codon sequences at single-base resolution (the mixed ratio was 1:1), ten times of the staple was added for each address in the medium DNA (the mixed ratio was 1: (number of addresses×10). The DNA mixture was denatured at 95° C. for 2 min, then annealed slowly by cooling down gradually to room temperature overnight.

### Example 3

This example presents a method of nanopore single-channel recording that is used in other examples.

Nanopore single-channel recording was conducted according to previously reported methods (Wang et al., 2011; Tian et al., 2018). Briefly, a lipid bilayer membrane (1,2-diphytanoyl-sn-glycero-3-phosphocholine) was formed over a 100-150 p.m orifice in the center of a Teflon film that partitioned between cis and trans recording solutions. Both solutions contained KCl (1 M) and were buffered with 10 mM Tris (pH 7.8). The MspA proteins were added to the cis solution, from which they inserted into the bilayer to form a single nanopore channel. The DNA chains were added to the cis solution. Voltage was applied to the trans solution while the cis solution was grounded. Ionic current through the pore was recorded using an Axopatch 200B amplifier, filtered with a built-in 4-pole low-pass Bessel Filter at 5 kHz, and acquired with Clampex 9.0 software through a Digidata 1440 A/D converter at a sampling rate of 20 kHz. Data of single-channel event amplitudes were analyzed by Clampfit 9.0, Excel, and SigmaPlot (SPSS) software. All measurements were conducted at 22±2° C. The program Frame-Shift Coding Window Finder was written by Python.

Examples 4-10 are directed to reading data encoded in a nucleic acid chain.

### Example 4

This examples presents the design of a coupled Frameshift encoding/Nanopore Unzip-Sequencing (NP Unzip-Seq) decoding workflow.

FIG. 1. Coupled Frameshift Encoding/NP Unzip-Seq Decoding strategy for data storage/retrieval from a universal DNA. Data was stored as a sequence of multi-nucleotide codons on a universal template and retrieved by reading the codon sequence in a nanopore. Each bit of the data was encoded by an antisense oligo staple. To write the data, all the staples were hybridized with the template at their addresses. The MspA nanopore recognized the first four unpaired bases of the template from the end of each template-staple duplex. An encoding mechanism was applied to write multinary datasets in the universal template. The universal template was a 1D array of data units. Each unit consisted of a coding window followed by an address. Staples of different lengths shifted the codon frame by different numbers of bases, generating multiple codons within the coding window at each address. This system provided model DNA hard drive that wrote and read different multinary target data in a universal template. Its workflow was as follows: A target dataset was uploaded to the cloud where it was converted to a digital sequence (i); Then a set of staples that encoded the digital sequence was collected from a synthetic staple library into a sample aliquot (ii); To write the data, the staple collection was mixed and hybridized with a universal template to store data as a codon sequence (iii); To retrieve the data, the staple-bound template was decoded by NP Unzip-Seq codon by codon (see SEQ ID NO: 1) (iv), which read all the codons from the electrical signatures (v); The codon sequence was decoded into digital sequence (vi), which was finally converted back to the data (vii) that was downloaded from the cloud.

### Example 5

This example presents a method for reading data stored in nucleic acid chains as codons.

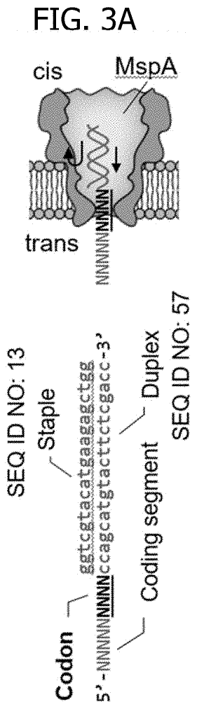

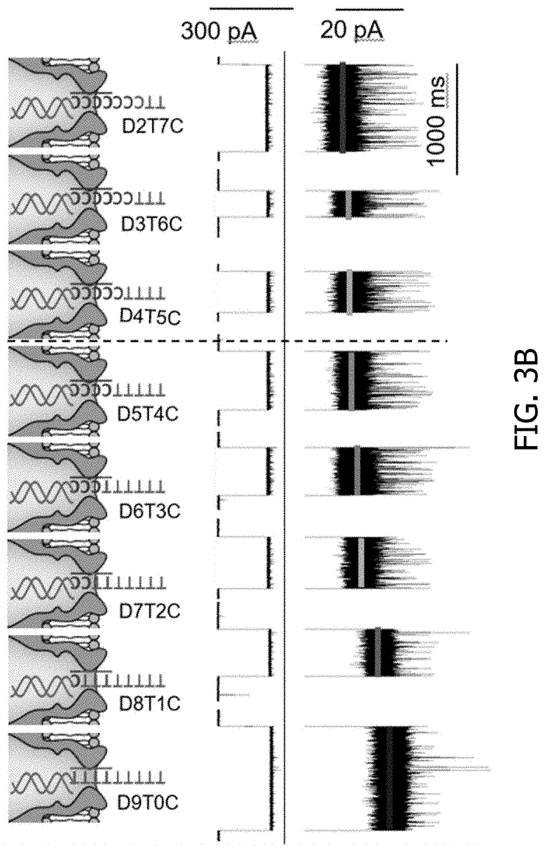

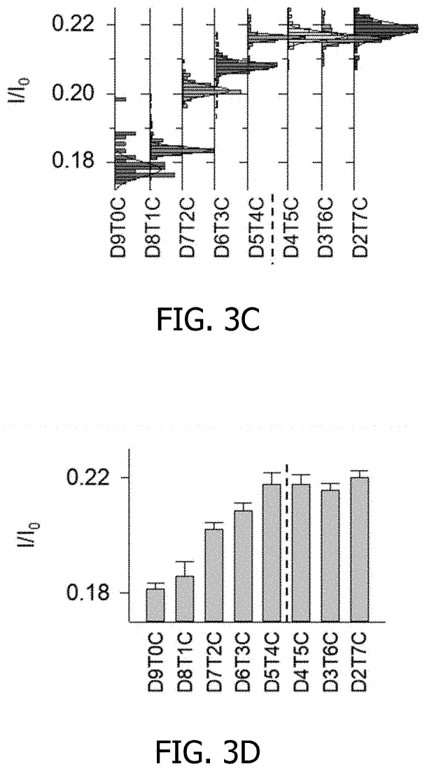

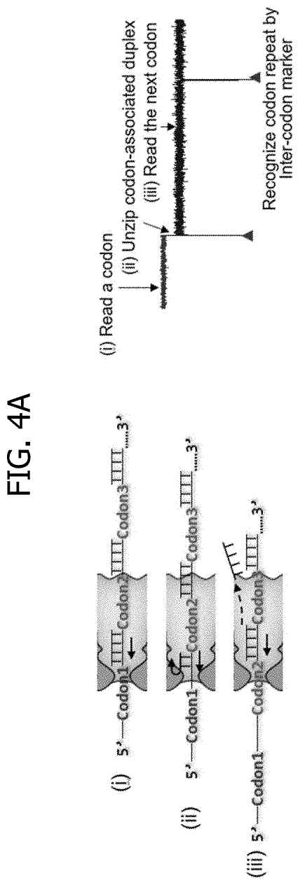

The nanopore was used to electrically screen a group of DNA coding segments with gradual nucleotide substitution. From D9T0C to D2T7C, one to seven thymines from the end of the duplex blocker were successively replaced by cytosines. These coding segments were extended with a common address, which bound a common staple (SEQ ID NO: 13) to form a double-stranded segment (FIG. 3A, Table 1). Table 1 is shown below for ease of reference.

TABLE 1
Sequences and blocking levels for studying nanopore discrimination of
codon sequences at single-nucleotide resolution.

| Name | SEQ ID NO: | Sequence | 1/10 | SD |
|---|---|---|---|---|
| D9T0C | 2 | TTTTTTTTTCCAGCATGTACTTCTCGACC | 0.18 | 0.0021 |
| D8T1C | 3 | TTTTTTTTCCCAGCATGTACTTCTCGACC | 0.1852 | 0.0052 |
| D7T2C | 4 | TTTTTTTCCCCAGCATGTACTTCTCGACC | 0.2012 | 0.0024 |
| D6T3C | 5 | TTTTTTCCCCCAGCATGTACTTCTCGACC | 0.2088 | 0.0027 |
| D5T4C | 6 | TTTTTCCCCCCAGCATGTACTTCTCGACC | 0.2167 | 0.0041 |
| D4T5C | 7 | TTTTCCCCCCCAGCATGTACTTCTCGACC | 0.2182 | 0.0033 |
| D3T6C | 8 | TTTCCCCCCCCAGCATGTACTTCTCGACC | 0.2164 | 0.0024 |
| D2T7C | 9 | TTCCCCCCCCCAGCATGTACTTCTCGACC | 0.2214 | 0.0025 |
| D1T8C | 10 | TCCCCCCCCCCAGCATGTACTTCTCGACC | | |
| D0T9C | 11 | CCCCCCCCCCCAGCATGTACTTCTCGACC | | |
| Staple11-FS | 12 | GGTCGAGAAGTACATGCTGG | | |

Driven by the voltage, each coding segment was pulled into the MspA nanopore from the cis opening, immobilized temporarily in the cavity by the double-stranded segment while characteristically regulating the nanopore ion current, and finally translocated through the pore once the blocker was unbound. This single-molecule procedure was recorded by the nanopore current signatures (FIG. 3B, +100 mV, 1 M KCl). The signature currents (FIG. 3C, 3D) showed that as the number of T→C substitutions increased from 0 (D9T0C) to (4) D5T4C, the blocking level (I/I₀) (Table 1) continuously and significantly increased from 18.0±2% for D9T0C, to 18.5±5% for D8T1C, 20.1±2% for D7Td2C, 20.9±3% for D6T3C, and 21.7±4% for D5T4C. Starting from D5T4C, more T→C substitutions in the coding segment no longer increased but retained the blocking level between 21.7-22.0%. This screening result indicated that the nanopore conductance was sensitive to the first four unpaired bases of the coding segment from the end of the duplex blocker. This screening result also indicated that the nanopore conductance was sensitive to changes to a single base in the codon of the coding segment (i.e. the nanopore was sensitive at single-base resolution).

Without being bound by particularly theory, it is believed that, when the duplex blocker was trapped into and anchored in the nanopore cavity, the first four bases connecting to the duplex exactly occupied the sharp end of the funnel-shaped MspA pore, i.e. the sequence-reading zone, whereas the remaining unpaired sequence was left out of the pore without influencing the nanopore conductance. Therefore, the first four bases directly connecting to (from the end of) the duplex blocker served as codons for data encoding. Codons were distinguished from each other by the signature pattern, including the blocking level, duration, noise, and other pattern characters.

FIGS. 3A-3D. Codon recognition at single-nucleotide resolution in the MspA nanopore. FIG. 3A. Structures of eight probing DNAs from D9T0C to D2T7C (Table 1 for sequences) and schematics of their configurations in the nanopore. The functional motifs, including coding segment, staple (SEQ ID NO: 13), duplex blocker and codons were labeled. The sequences of quadromeric (4 bases) codons that were discriminated by the nanopore were bolded, which are located from the end of the duplex blocker; FIG. 3B. Representative nanopore current traces showing the blockades (top) and enlarged blockades (bottom) for all the eight probing DNAs, recorded at +100 mV (cis grounded) with 1 M KCl in both cis and trans solution; c. Histograms (left) and values (right) of the signature blocking levels (I/I₀) as the function of the number of T→C substitutions for all the probing DNAs. The vertical dash line separates all the DNAs into two groups with different dependence of the nanopore current to the number of T→C substitutions, leading to the finding that quadromeric codons that were discriminated by the nanopore at the single-nucleotide resolution and used for data encoding.

### Example 6

This example shows design of codon sequences for studying reading sequential data codons and discriminating consecutive identical codons.

A group of DNA templates were designed with three-quadromeric codons. These templates, from D000 through D111, encoded all the 3-bit binary numbers, i.e. 000, 001, 010, 011, 100, 101, 110, and 111 (FIG. 4B, Table 2). Table 2 is shown below for ease of reference.

TABLE 2
Sequences and blocking levels for studying nanopore
discrimination of sequential codon sequences.

| | SEQ | . . . Codon1 . . . | Codon | Codon | Codon | Codon | Codon | Codon |
|---|---|---|---|---|---|---|---|---|
| | ID | Codon2 . . . | Codon 1 | 1 | 2 | 2 | 3 | 3 |
| | NO: | Codon3 . . . | 1/10 | SD | 1/10 | SD | 1/10 | SD |
| D000 | 14 | CCCCCCCCCCCCAGCATGTACTTCTCGACCC | 0.18 | 0.0021 | 0.2551 | 0.0078 | 0.2575 | 0.002 |
| | | CCCCCCAGCATGTACTTCTCGACCC | | | | | | |
| | | CCCCCCAGCATGTACTTCTCGACC | | | | | | |
| D001 | 15 | CCCCCCCCCCCCAGCATGTACTTCTCGACCC | 0.1852 | 0.0052 | 0.2502 | 0.0058 | 0.2289 | 0.0058 |
| | | CCCCCCAGCATGTACTTCTCGACCT | | | | | | |
| | | TTTTCCAGCATGTACTTCTCGACC | | | | | | |
| D010 | 16 | CCCCCCCCCCCCAGCATGTACTTCTCGACCT | 0.2012 | 0.0024 | 0.2136 | 0.0053 | 0.2618 | 0.0071 |
| | | TTTTCCAGCATGTACTTCTCGACCC | | | | | | |
| | | CCCCCCAGCATGTACTTCTCGACC | | | | | | |
| D011 | 17 | CCCCCCCCCCCCAGCATGTACTTCTCGACCT | 0.2088 | 0.0027 | 0.2164 | 0.005 | 0.2224 | 0.0024 |
| | | TTTTCCAGCATGTACTTCTCGACCT | | | | | | |
| | | TTTTCCAGCATGTACTTCTCGACC | | | | | | |
| D100 | 18 | CCCCCTTTTTCCAGCATGTACTTCTCGACCC | 0.2167 | 0.0041 | 0.2538 | 0.0065 | 0.2642 | 0.0028 |
| | | CCCCCCAGCATGTACTTCTCGACCC | | | | | | |
| | | CCCCCCAGCATGTACTTCTCGACC | | | | | | |
| D101 | 19 | CCCCCTTTTTCCAGCATGTACTTCTCGACCC | 0.2182 | 0.0033 | 0.2525 | 0.004 | 0.2252 | 0.0043 |
| | | CCCCCCAGCATGTACTTCTCGACCT | | | | | | |
| | | TTTTCCAGCATGTACTTCTCGACC | | | | | | |
| D110 | 20 | CCCCCTTTTTCCAGCATGTACTTCTCGACCC | 0.2164 | 0.0024 | 0.2148 | 0.0054 | 0.2518 | 0.0036 |
| | | TTTTCCAGCATGTACTTCTCGACCC | | | | | | |
| | | CCCCCCAGCATGTACTTCTCGACC | | | | | | |
| D111 | 21 | CCCCCTTTTTCCAGCATGTACTTCTCGACCT | 0.2214 | 0.0025 | 0.2184 | 0.0057 | 0.2229 | 0.0045 |
| | | TTTTCCAGCATGTACTTCTCGACCT | | | | | | |
| | | TTTTCCAGCATGTACTTCTCGACC | | | | | | |
| Staple11- | 12 | GGTCGAGAAGTACATGCTGG | | | | | | |
| FS | | | | | | | | |

Two nanopore-discriminable codons, CCCC and TTTT (FIG. 3A, FIG. 3B), were selected to represent binary values 0 and 1. All codons were associated with a common templatestaple duplex. Thus, each DNA formed a tandem codon-duplex complex.

FIGS. 4A-4C. Sequentially reading codons and discriminating codon repeats in a template DNA by NP Unzip-Seq. FIG. 4A. Conformations and nanopore signatures for the three steps in codon reading, (i) discriminated the identity of a codon from the signature that is immobilized in the sequence-reading zone of the MspA pore, (ii) produced inter-codon marker when the codon-associated duplex blocker is unzipped, and (iii) pulled the next codon into the nanopore for reading its identity; FIG. 4B. Sequentially reading 3-bit binary codons. (Upper) Structure of the 3-codon template. Each codon was associated with a common duplex blocker formed by hybridization with a common staple. (Lower) Schematics and nanopore current signatures for reading all the eight 3-bit binary sequences (000, 001, 010, 011, 100, 101, 110 and 111). Two codons CCCC and TTTT were selected to encode binary values 0 and 1. Their current levels in the signature were marked by lines, and the inter-codon markers were marked by triangles, which separate consecutive identical codons for 00, 11, 000 and 111 that share identical current levels; FIG. 4C Reading a long codon template by NP Unzip-Seq. (Upper) Structure of the 8-codon template (D8) that encodes the 8-bit binary ASCII code of the letter ‘M’ (01001101). (Lower) Schematic and nanopore current signature for reading 01001101 by NP Unzip-Seq. Consecutive identical codons for 00 and 11 that share identical current levels were separated by inter-codon markers (triangles).

### Example 7

This example shows reading the sequences of Example 6.

Since D010 and D101 did not contain codon repeats, their codons were directly read out from the blocking levels. Their nanopore signatures were separated into three stages with sequential blocking levels (I/I₀) at 25.7±0.6% m(high)/21.4±0.5% (low)/26.2±0.7% (high) for D010, and 21.6±0.2% (low)/25.3±0.4% (high)/22.5±0.4% (low) for D101. According to the fact that the blocking level for CCCC was higher than TTTT (FIG. 3A, 3B), the three stages in D010's signature were assigned to CCCC (Codon 1), TTTT (Codon 2), and CCCC (Codon 3), accurately reading out the binary number 010. Similarly, the three stages in D101's signature were assigned to TTTT (Codon 1), CCCC (Codon 2), and TTTT (Codon 3), accurately outputting binary 101. In contrast to D010 and D101, all other six DNAs contained consecutive identical codons for 00, 11, 000, or 111. Their identical blocking levels did not distinguish these repeat codons.

### Example 8

This example presents evidence of inter-codon markers for use in demarcating codon stops and starts, particularly between codon repeats

When re-examining the D010 and D101's signatures, two downward current flicks were identified with distinctively lower conductance at Stage 1/2 and Stage 2/3 transitions (marked by triangles). These ‘inter-codon markers’ recognized the end of one codon signal and the beginning of the next codon signal, therefore becoming another codon identifier in addition to the blocking level. The signatures for all the six DNAs containing consecutive identical codons exhibited two inter-codon markers (FIG. 4B) that separated the three codons, therefore confirming that such an ‘inter-codon marker’ is a universal identifier that can be used to separate consecutive identical codons, despite their identical blocking level in the signature. For example, the inter-codon markers were identified to separate D011's double TTTTs (Codon 2/3) for 11, D000's triple CCCCs for 000, and D111's triple TTTTs for 111, thus accurately decoding 011, 000, and 111. Such important electric markers could be generated by the staple, which remains trapped in the nanopore after fully or partially unzipped from the template, co-occupying the nanopore lumen with the next duplex; or by the template DNA, which is grasped by the highly positively charged domain of the MspA pore during transient unzipping, causing the unzipped part of the template to ‘curl’ when pushed by the applied voltage.

### Example 9

This example presents design of barcodes using the 3-bit system.

Because these stapled DNA were easily and unmistakably read out by the nanopore, they could be linked as a barcode module to eight different probes/receptors (via synthesis or conjugation) to simultaneously detect eight biomarkers. It uses the values of three binary bits to form eight barcodes, 000, 001, 010, 011, 100, 101, 110, and 111. Their resulting nanopore electric signatures included two modular components, a barcode signal followed by a biomarker binding signal. By reading the barcode signals, which biomarker was being detected was able to be discriminated, and by reading the biomarker signal, if the probe/receptor is bound or not bound by its target biomarker was able to be identified.

### Example 10

This example shows how NP-Unzip-Seq is used to read long DNAs that encodes the 8-bit binary ASCII code of the letter ‘M.’

To test the capability in reading long DNAs, a 200-nt template (D8) was designed that uses CCCC (0) and TTTT (1) to encode the eight-bit binary sequence 01001101 for the letter ‘M’ (FIG. 4C). A start and an ending codon were added to simulate the bookmark of the data region. Again, all codons were associated with a common templatestaple duplex (Table 3). Table 3 is shown below for ease of reference.

TABLE 3
Sequences and blocking levels for 8-bit binary encoding.

| | D01001101 (D8) | Staple3 |
|---|---|---|
| . . . Start . . . Codon1 . . . | CGATGCCTGCTGCTCTGACCCCCCCCCTGCTGCTCTGACCT | GGTCAGAGCAGCAGG |
| Codon2 . . . Codon3 . . . | TTTTCCTGCTGCTCTGACCCCCCCCCTGCTGCTCTGACCC | |
| Codon4 . . . Codon5 . . . | CCCCCCTGCTGCTCTGACCTTTTTCCTGCTGCTCTGACCT | |
| Codon6 . . . Codon7 . . . | TTTTCCTGCTGCTCTGACCTCCCCCCTGCTGCTCTGACCT | |
| Codon8 . . . Stop . . . | TTTTCCTGCTGCTCTGACCCGATGCCTGCTGCTCTGACC | |
| SEQ ID NO: | 22 | 23 |
| Codon 1 1/10 | 0.2472 | |
| Codon 1 SD | 0.0042 | |
| Codon 2 1/10 | 0.2058 | |
| Codon 2 SD | 0.0074 | |
| Codon 3 1/10 | 0.2489 | |
| Codon 3 SD | 0.004 | |
| Codon 4 1/10 | 0.2485 | |
| Codon 4 SD | 0.0006 | |
| Codon 5 1/10 | 0.1993 | |
| Codon 5 SD | 0.004 | |
| Codon 6 1/10 | 0.1984 | |
| Codon 6 SD | 0.0087 | |
| Codon 7 1/10 | 0.2424 | |
| Codon 7 SD | 0.0067 | |
| Codon 8 1/10 | 0.1984 | |
| Codon 8 SD | 0.0036 | |

The nanopore signatures (FIG. 4C) revealed the entire procedure for vectorial unzipping of this tandem codon-duplex complex. Except for the initial and terminal current patterns for the start and end codons, two main blocking levels were identified in the data region, which ranged between 24.2±0.6%-24.9±0.4% for codon CCCC and 19.8±0.4%-20.6±0.7% for codon TTTT. The two current levels separated the data region into 6 stages for Codon 1 (CCCC, 0), Codon 2 (TTTT, 1), Codon 3/Codon 4 (CCCC, 00), Codon 5/Codon 6 (TTTT, 11), Codon 7 (CCCC, 0) and Codon 8 (TTTT, 1). Identical codons in Codon 3/Codon 4 and Codon 5/Codon 6 could not be discriminated from their identical blocking levels but were separated into two stages by the inter-codon markers. Thus, Codon 3/Codon4 (CCCC, 00) and Codon 5/Codon 6 (TTTT, 11) were accurately read out. Overall, NP Unzip-Seq discriminated all the eight codons, including two codon repeats in the template, accurately decoding all eight bits of 01001101, outputting the letter ‘M’.

### Example 11

This example presents a method for encoding data and reading the encoded data stored in codons in nucleic acid chains via blockers that bind to address sections, encoders that allow for frameshift encoding, and nanopore sequencing.

TTTTCCCCC was used as the coding window to exemplify the Frameshift Encoding process in a universal template (FIG. 5A, 5B). According to the principle (FIG. 1, FIG. 5A), a universal template was designed with 3-data units for writing various 3-bit binary data (FIG. 5B). Each data unit i possessed a common coding window TTTTCCCCC and address i that was bound by a pair of staples, Staple_{i1-FS} and Staple_{i2-FS} (Table 4). Table 4 is shown below for ease of reference.

TABLE 4
Frameshift (FS) coding windows, codon addresses, staples, and codons
for 3-bit frameshift encoding.

| | | Frameshift Coding Window-Codon1 | | |
|---|---|---|---|---|
| | | Address- | | |
| | | Frameshift Coding Window-Codon2 | | |
| | | Address- | | |
| Sequence | SEQ | Frameshift Coding Window-Codon3 | | |
| Names | ID NO: | Address | Codon Name | Form |
| D-FS | | TTTTTCCCCCCCAGCATGTACTTCTCGACC | | |
| | | TTTTTCCCCCGGATTTCAAGTTCTCCCTCC | | |
| | | TTTTTCCCCCGCTCTTCAAGGTGCACATGG | | |
| Staple11-FS | 24 | GGTCGAGAAGTACATGCTGG | Codon11 | CCCC |
| Staple12-FS | 25 | GGTCGAGAAGTACATGCTGGGGGGG | Codon12 | TTTT |
| Staple21-FS | 26 | GGAGGGAGAACTTGAAATCC | Codon21 | CCCC |
| Staple22-FS | 27 | GGAGGGAGAACTTGAAATCCGGGGG | Codon22 | TTTT |
| Staple31-FS | 28 | CCATGTGCACCTTGAAGAGC | Codon31 | CCCC |
| Staple32-FS | 29 | CCATGTGCACCTTGAAGAGCGGGGG | Codon32 | TTTT |

Upon binding to address i, Staple_{i1-FS}'s encoder was null, thus did not block the coding window, allowing its last four bases CCCC to form Codonᵢ₁; Staple_{i2-FS} had a GGGGG encoder, which blocked the last five bases CCCCC of the coding window, therefore upstream shifting the codon frame by 5 bases (−5 frameshift) to form another codon TTTT. As a result, the staple encoders enabled the same coding window to form two nanopore-discriminable codons, CCCC and TTTT, which allowed for freely ‘writing’ either ‘0’ or ‘1’ at each address, and thereby storing various binary datasets in the universal template.

To write 000, Staple_{11-FS}/Staple_{21-FS}/Staple_{31-FS} (SEQ ID NOs: 12, 26, and 28) were selected and this staple panel was mixed with the template. To write 001, Staple_{31-FS} (SEQ ID NO: 28) was replaced with Staple_{32-FS} (SEQ ID NO: 29) and the panel of Staple_{11-FS}/Staple_{21-FS}/Staple_{32-FS} (SEQ ID NOs: 12, 26, and 29) was mixed with the same template. Similarly, to write 010 and 011, the panels of Staple_{11-FS}/Staple_{22-FS}/Staple_{31-FS} (SEQ ID NOs: 12, 27, and 28) and Staple_{11-FS}/ Staple_{22-FS}/Staple_{32-FS} (SEQ ID NOs: 12, 27, and 29) were used, and again, mixed with the same template, respectively. The nanopore signatures for all the four tandem codon-templatestaple duplex complexes (FIG. 5B) were separated into three stages by the inter-codon markers, showing that all the DNA⋅Staple complexes underwent three sequential unzipping procedures, and each unzipping step decoded one bit of the 3-bit binary numbers. The three stages in each signature were found at one of two blocking levels ranging between 22.5±0.8%-24.2±0.6% and 18.4±0.6%-19.4±0.2%, consistent with that produced by the codons CCCC and TTTT (Table 5). Table 5 is shown below for ease of reference.

TABLE 5
Blocking levels for 3-bit frameshift (FS) encoding.

| | Codon1 | | Codon2 | | Codon3 | |
|---|---|---|---|---|---|---|
| | I/I0 | SD | I/I0 | SD | I/I0 | SD |
| D000 (FS) | 0.2339 | 0.0076 | 0.2254 | 0.0075 | 0.2314 | 0.0019 |
| D001(FS) | 0.2327 | 0.0083 | 0.2255 | 0.0016 | 0.1907 | 0.0028 |
| D010 (FS) | 0.2418 | 0.0054 | 0.1944 | 0.0019 | 0.2381 | 0.0022 |
| D011 (FS) | 0.2314 | 0.0085 | 0.1838 | 0.0064 | 0.1909 | 0.0059 |

Thus, each stage in the signature was assigned to either codon CCCC for ‘0’ or TTTT for ‘1’. With these codon assignments, the four signatures accurately output the binary numbers 000, 001, 010, and 011, proving that the universal template was translated into different codon sequences for storing different datasets.

In summary, for the first time a frameshift strategy for encoding different data into a universal template was shown, establishing a model of DNA hard drive capable of rapid, synthesis-free data writing, retrieval, and rewriting.

FIGS. 5A and 5B. Writing different binary datasets in a universal template by frameshift encoding and reading data by NP Unzip-Seq. FIG. 5A. Frameshift encoding. (Left) A universal media DNA was designed that uses a series of units to store data. Each unit consisted of a Coding Window and an address. The same sequence was used as a coding window for the i^{th} Unit (see SEQ ID NO: 30). (Right) Diagram showing binary encoding by frameshift in a universal template. Staple_{i1-FS} and Staple_{i2-FS} carried 0 (null), and 5 guanine (GGGGG) encoders at the 3′ end. Upon binding to Addressᵢ, their encoders ‘silenced’ 0 and 5 cytosine (CCCCC) in the coding window, shifting the codon frame by 0 and 5 bases to form codons CCCC and TTTTT, respectively. To store a dataset, a panel of staples was selected to form data-specific codons in the universal DNA; FIG. 5B. Schematics for writing 3-bit binary numbers, 000, 001, 010, and 011, in a universal DNA by using different panels of staples (Table 4 for sequences), and nanopore current signatures for reading these numbers. The three units used the same coding window but different address sequences. Codons CCCC and TTTT were used for 0 and 1. The ‘silenced’ sequences by encoders in the coding window were underlined, and the inter-codon markers were marked by triangles.

Examples 12-15 are directed to frameshift encoding and decoding multinary data in native DNA sequences.

### Example 12

This example presents a method for frameshift encoding data and reading the encoded data stored in native sequences via blockers that bind to address sections, encoders that allow for frameshift encoding, and nanopore sequencing.

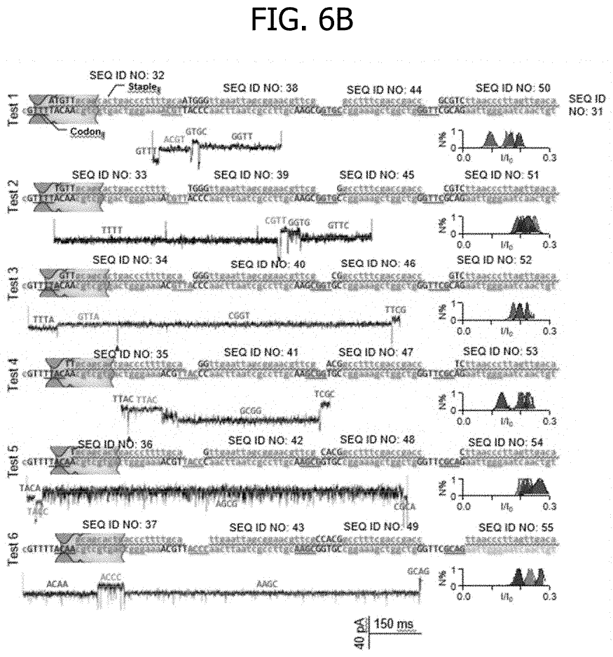

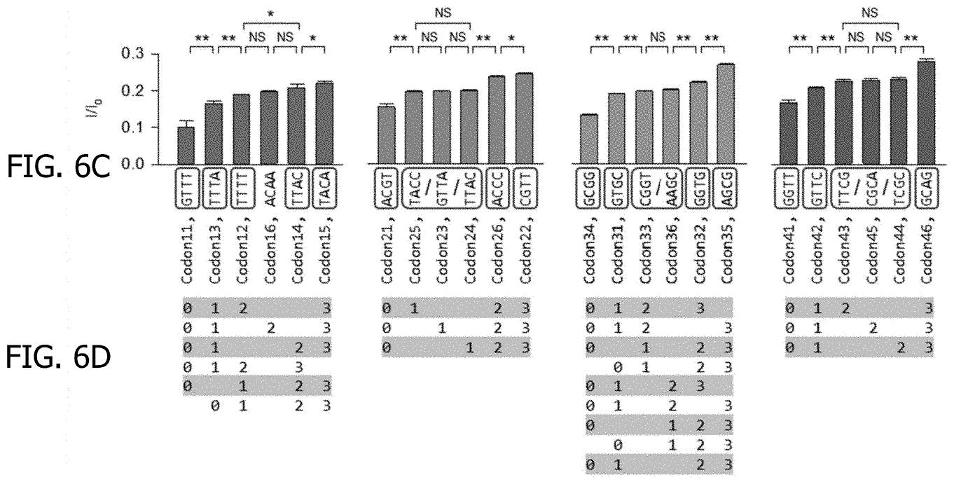

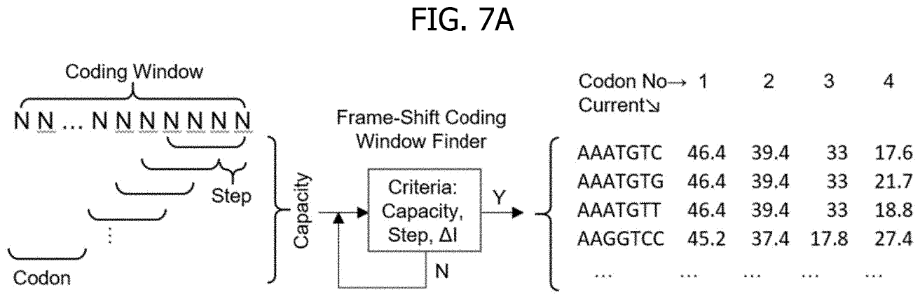

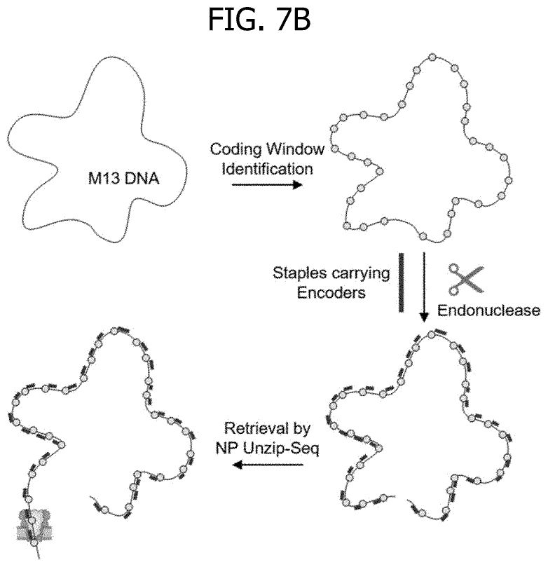

Frameshift Encoding/NP Unzip-Seq decoding of multinary data using sequences from native DNAs was validated. Four different segments from the M13mp18 DNA were randomly truncated, a popular template for DNA origami construction (FIG. 6A), to represent four data units, and combined them into a single template. Each data unit contained a unique 9-nt coding window and a 14˜17-nt address. The nine nucleotides in a coding window formed a total of six different quadromeric codons by 1-nt step frameshifting with a panel of 6 staples, Stapleᵢ₁ through Stapleᵢ₆ (i=1-4 represents the i^{th} coding window) (Table 6). Table 6 is shown below for ease of reference.

TABLE 6
Sequences, frameshift coding windows, addresses, codons, and staples for native sequence
encoding with non-labeled bolded sequences as encoders.

| | | . . . Frameshift Coding Window . . . Address1 . . . Frameshift | | |
|---|---|---|---|---|
| Sequence | SEQ | Coding Window . . . Address2 . . . Frameshift Codong Window . . . | Codon | |
| Name | ID NO: | Address3 . . . Frameshift Coding Window . . . Address4 | Name | Form |
| M13-Temp | 31 | cGTTTTACAAcgtcgtgactgggaaaACGTTACCCaacttaatcgccttgc | | |
| | | AAGCGGTGCcggaaagctggctgGGTTCGCAGaattgggaatcaactgt | | |
| Staple11 | 32 | ACGTTTTCCCAGTCACGACGTTGTA | Codon11 | GTTT |
| Staple12 | 33 | TTTTCCCAGTCACGACGTTGT | Codon12 | TTTT |
| Staple13 | 34 | AACGTTTTCCCAGTCACGACGTTG | Codon13 | TTTA |
| Staple14 | 35 | AACGTTTTCCCAGTCACGACGTT | Codon14 | TTAC |
| Staple15 | 36 | AACGTTTTCCCAGTCACGACGT | Codon15 | TACA |
| Staple16 | 37 | ACGTTTTCCCAGTCACGACG | Codon16 | ACAA |
| Staple21 | 38 | GCTTGCAAGGCGATTAAGTTGGGTA | Codon21 | ACGT |
| Staple22 | 39 | GCTTGCAAGGCGATTAAGTTGGGT | Codon22 | CGTT |
| Staple23 | 40 | GCTTGCAAGGCGATTAAGTTGGG | Codon23 | GTTA |
| Staple24 | 41 | GCTTGCAAGGCGATTAAGTTGG | Codon24 | TTAC |
| Staple25 | 42 | GCTTGCAAGGCGATTAAGTTG | Codon25 | TACC |
| Staple26 | 43 | GCTTGCAAGGCGATTAAGTT | Codon26 | ACCC |
| Staple31 | 44 | CCAGCCAGCTTTCCG | Codon31 | GTGC |
| Staple32 | 45 | CCAGCCAGCTTTCCGG | Codon32 | GGTG |
| Staple33 | 46 | CCAGCCAGCTTTCCGGC | Codon33 | CGGT |
| Staple34 | 47 | CCAGCCAGCTTTCCGGCA | Codon34 | GCGG |
| Staple35 | 48 | CCAGCCAGCTTTCCGGCAC | Codon35 | AGCG |
| Staple36 | 49 | CCAGCCAGCTTTCCGGCACC | Codon36 | AAGC |
| Staple41 | 50 | ACAGTTGATTCCCAATTCTGCG | Codon41 | GGTT |
| Staple42 | 51 | ACAGTTGATTCCCAATTCTGC | Codon42 | GTTC |
| Staple43 | 52 | ACAGTTGATTCCCAATTCTG | Codon43 | TTCG |
| Staple44 | 53 | ACAGTTGATTCCCAATTCT | Codon44 | TCGC |
| Staple45 | 54 | ACAGTTGATTCCCAATTC | Codon45 | CGCA |
| Staple46 | 55 | ACAGTTGATTCCCAATT | Codon46 | GCAG |

The goal was to identify four distinguishable codons among six by NP Unzip-Seq to realize quaternary data encoding. A total of 4×6 different codons formed by their staples were detected in six tests (Table 7).

TABLE 7
Blocking levels for native sequence encoding for 6 codons formed by
Coding Window i for Bit i.

| | Testj | | | | | | |
|---|---|---|---|---|---|---|---|
| | (j = 1-6) | Test 1 | Test 2 | Test 3 | Test 4 | Test 5 | Test 6 |
| 6 codons in | Codon 1# | Codon11 | Codon12 | Codon13 | Codon14 | Codon15 | Codon16 |
| Coding | Codon | GTTT | TTTT | TTTA | TTAC | TACA | ACAA |
| Window 1 | 1/10 | 0.101 | 0.188 | 0.165 | 0.207 | 0.221 | 0.197 |
| for Bit 1 | SD | 0.0168 | 0.0021 | 0.0075 | 0.0099 | 0.0048 | 0.0044 |
| 6 codons in | Codon2# | Codon21 | Codon22 | Codon23 | Codon24 | Codon25 | Codon26 |
| Coding | Codon | ACGT | CGTT | GTTA | TTAC | TACC | ACCC |
| Window 2 | 1/10 | 0.155 | 0.246 | 0.198 | 0.199 | 0.197 | 0.238 |
| for Bit 2 | SD | 0.0108 | 0.0024 | 0.0018 | 0.0035 | 0.0035 | 0.0021 |
| 6 codons in | Codon3# | Codon31 | Codon32 | Codon33 | Codon34 | Codon35 | Codon36 |
| Coding | Codon | GTGC | GGTG | CGGT | GCGG | AGCG | AAGC |
| Window 3 | 1/10 | 0.191 | 0.223 | 0.196 | 0.134 | 0.27 | 0.201 |
| for Bit 3 | SD | 0.0025 | 0.0024 | 0.0037 | 0.0032 | 0.003 | 0.0043 |
| 6 codons in | Codon4# | Codon41 | Codon42 | Codon43 | Codon44 | Codon45 | Codon46 |
| Coding | Codon | GGTT | GTTC | TTCG | TCGC | CGCA | GCAG |
| Window 4 | 1/10 | 0.167 | 0.206 | 0.226 | 0.23 | 0.228 | 0.279 |
| for Bit 4 | SD | 0.0066 | 0.0041 | 0.005 | 0.0054 | 0.0047 | 0.0087 |

In each test, four staples were selected, each from a panel, to sequentially ‘write’ four codons in the template, equivalent to encoding a 4-bit quaternary data. For example, in Test 1, Staple₁₁ (SEQ ID NO: 32), Staple₂₁ (SEQ ID NO: 38), Staple₃₁ (SEQ ID NO: 44) and Staple₄₁ (SEQ ID NO: 50) were used to generate Codon₁₁ (GTTT), Codon₂₁ (ACGT), Codon₃₁ (GTGC) and Codon₄₁ (GGTT), and in Test 2, Staple₁₂ (SEQ ID NO: 33), Staple₂₂ (SEQ ID NO: 39), Staple₃₂ (SEQ ID NO: 45) and Staple₄₂ (SEQ ID NO: 51) were used to generate Codon₁₂ (TTTT), Codon₂₂ (GCTT), Codon₃₂ (GGTG) and Codon₄₂ (GTTC). For coding windows 1, 2, and 4, the six staples in the panel were tested in the order of +1 (5′→3′ 1-nt step) frameshift. For example, Staple₁₁ used the ATGTT to generate Codon₁₁ (GTTT). From Staple₁₂ to Staple₁₆ (SEQ ID NOs: 33-37), their encoders were successively shortened by one base from the 3′ end to TGTT, GTT, TT, T, and null. As a result, the codon frame shifted base by base in the 5′→3′ direction to generate five new codons Codon₁₂ through Codon₁₆ (TTTT, TTTA, TTAC, TACA, and ACAA). For coding window 3, the six staples in the panel were tested in the order of −1 (3′→5′ 1-nt step) frameshift.

The nanopore current signatures for all the six template⋅staples complexes revealed four stages, as identified from their characteristic signature patterns, including blocking levels and noise, and/or inter-codon markers (FIG. 6B and Table 7).

In Test 1, the nanopore signature showed four stages that were separated by the blocking levels, 10.1±1.7%, 15.5±1.1%, 19.1±0.3%, and 16.7±0.7%, suggesting that the nanopore sequentially read the four different codons, Codon₁₁ (GTTT), Codon₂₁ (ACGT), Codon₃₁ (GTGC), and Codon₄₁ (GGTT) formed in the template. Similar findings were also obtained from Test 2 and Test 4-6. The Test 3's signature only revealed three stages based on the blocking levels, with the first and last stages at 16.5±0.8% and 22.6±0.1%. However, the middle stage was split by an inter-strand marker (marked by triangle) into two separate stages with identical blocking levels at 19.8±0.2% and 19.6±0.4%. Therefore, again, the universal inter-strand marker was proven to be a powerful codon identifier, which was jointly used with the blocking levels to accurately identify the four stages. This result demonstrated the writing of the four sequential codons, Codon₁₃ (TTTA), Codon₂₃ (GTTA), Codon₃₃ (CGGT), and Codon₄₃ (TTCG) in the template. Overall, the four stages in all the six signatures were assigned to the four sequential codons, confirming the capability of NP-Unzip-Seq in a sequential reading of various codons in the template by unzipping of each template⋅staple duplex. In conclusion, all the four 9-nt coding windows in the universal template were proven to be able to generate a panel of six different codons by Frameshift Encoding.

FIGS. 6A-6D. Frameshift encoding/NP Unzip-Seq decoding of multinary data in a sequence from native DNA. FIG. 6A. Sequences of four data unit segments randomly truncated from the M13 DNA to form a universal template. Each data unit contained a coding window followed by an address sequence; FIG. 6B. Sequences of the template in complex with staples showing the formation of multiple codons in each coding window by staple-facilitated frameshifting base by base, and nanopore current signature showing sequential readout of the four codons by NP Unzip-Seq. The currents for four sequential codons were marked by lines. Current amplitude histograms were shown for extracting the average blocking levels for the four codons; FIG. 6C. Blocking levels (I/I₀) for the six codons that were formed in each coding window by frameshifting for the four coding windows. Codons were arranged according to the blocking level from low to high. All the codon pairs among six were analyzed by Tukey's multiple comparison test to rank their nanopore discrimination capability as highly discriminable (**, p<0.001), discriminable (*, p<0.05), and indiscriminate (NS, not significant). Note that all the codon pair comparisons not shown in the plot were highly discriminable; FIG. 6D. Selection of four nanopore-discriminable codons among six in each coding window to form various quartet codon panels for quaternary data encoding. The four codons represented quaternary values 0, 1, 2, and 3.

### Example 13

This example presents statistics to show how codons are discriminable.

The blocking levels (I/I₀) of all the six codons that were written in a coding window were presented in the order of blocking level from low to high (FIG. 6C and Table 8). Table 8 is shown below for ease of reference.

TABLE 8
Blocking levels for native sequence encoding for 6 codons formed by Coding
Window i for Bit i shown in the order of the blocking level from low to high.

| 6 codons in | Codon 1# | Codon11 | Codon13 | Codon12 | Codon16 | Codon14 | Codon15 |
|---|---|---|---|---|---|---|---|
| Coding | Codon | GTTT | TTTA | TTTT | ACAA | TTAC | TACA |
| Window 1 | 1/10 | 0.101 | 0.165 | 0.188 | 0.197 | 0.207 | 0.221 |
| for Bit 1 | SD | 0.0168 | 0.00746 | 0.00205 | 0.00442 | 0.00993 | 0.00484 |
| 6 codons in | Codon2# | Codon21 | Codon25 | Codon23 | Codon24 | Codon26 | Codon22 |
| Coding | Codon | ACGT | TACC | GTTA | TTAC | ACCC | CGTT |
| Window 2 | 1/10 | 0.155 | 0.197 | 0.198 | 0.199 | 0.238 | 0.246 |
| for Bit 2 | SD | 0.0108 | 0.0035 | 0.0018 | 0.0035 | 0.0021 | 0.0024 |
| 6 codons in | Codon3# | Codon34 | Codon31 | Codon33 | Codon36 | Codon32 | Codon35 |
| Coding | Codon | GCGG | GTGC | CGGT | AAGC | GGTG | AGCG |
| Window 3 | 1/10 | 0.134 | 0.191 | 0.196 | 0.201 | 0.223 | 0.27 |
| for Bit 3 | SD | 0.0032 | 0.0025 | 0.0037 | 0.0043 | 0.0024 | 0.003 |
| 6 codons in | Codon4# | Codon41 | Codon42 | Codon43 | Codon45 | Codon44 | Codon46 |
| Coding | Codon | GGTT | GTTC | TTCG | CGCA | TCGC | GCAG |
| Window 4 | 1/10 | 0.167 | 0.206 | 0.226 | 0.228 | 0.23 | 0.279 |
| for Bit 4 | SD | 0.0066 | 0.0041 | 0.005 | 0.0047 | 0.0054 | 0.0087 |

All pairs of codons were analyzed by Tukey's multiple comparison test to determine their discrimination capability and were ranked as highly discriminable (p<0.001), discriminable (p<0.05), and indiscriminate (NS, not significant) (FIG. 6C and Table 9). Table 9 is shown below for ease of reference.

TABLE 9
All Pairwise Multiple Comparisons (Tukey Tests) of codon blocking levels for ranking
nanopore codon discrimination capability (Ranks of nanopore codon pair
discrimination capability: P < 0.001, **, highly discriminable; P < 0.05, *,
discriminable; and P > 0.05, NS (not significant),).

| 6 codons in Coding Window 1 | | | | 6 codons in Coding Window 2 | | | |
|---|---|---|---|---|---|---|---|
| Comparison | P | P < 0.050 | Rank | Comparison | P | P < 0.050 | Rank |
| TACA vs. GTTT | <0.001 | Yes | ** | CGTT vs. ACGT | <0.001 | Yes | ** |
| TACA vs. TTTA | <0.001 | Yes | ** | CGTT vs. TACC | <0.001 | Yes | ** |
| TACA vs. TTTT | <0.001 | Yes | ** | CGTT vs. GTTA | <0.001 | Yes | ** |
| TACA vs. ACAA | <0.001 | Yes | ** | CGTT vs. TTAC | <0.001 | Yes | ** |
| TACA vs. TTAC | 0.04 | Yes | * | CGTT vs. ACCC | 0.015 | Yes | * |
| TTAC vs. GTTT | <0.001 | Yes | ** | ACCC vs. ACGT | <0.001 | Yes | ** |
| TTAC vs. TTTA | <0.001 | Yes | ** | ACCC vs. TACC | <0.001 | Yes | ** |
| TTAC vs. TTTT | 0.001 | Yes | * | ACCC vs. GTTA | <0.001 | Yes | ** |
| TTAC vs. ACAA | 0.175 | No | NS | ACCC vs. TTAC | <0.001 | Yes | ** |
| ACAA vs. GTTT | <0.001 | Yes | ** | TTAC vs. ACGT | <0.001 | Yes | ** |
| ACAA vs. TTTA | <0.001 | Yes | ** | TTAC vs. TACC | 0.939 | No | NS |
| ACAA vs. TTTT | 0.322 | No | NS | TTAC vs. GTTA | 0.996 | No | NS |
| TTTT vs. GTTT | <0.001 | Yes | ** | GTTA vs. ACGT | <0.001 | Yes | ** |
| TTTT vs. TTTA | <0.001 | Yes | ** | GTTA vs. TACC | 0.999 | No | NS |
| TTTA vs. GTTT | <0.001 | Yes | ** | TACC vs. ACGT | <0.001 | Yes | ** |
| AGCG vs. GCGG | <0.001 | Yes | ** | GCAG vs. GGTT | <0.001 | Yes | ** |
| AGCG vs. GTGC | <0.001 | Yes | ** | GCAG vs. GTTC | <0.001 | Yes | ** |
| AGCG vs. CGGT | <0.001 | Yes | ** | GCAG vs. TTCG | <0.001 | Yes | ** |
| AGCG vs. AAGC | <0.001 | Yes | ** | GCAG vs. CGCA | <0.001 | Yes | ** |
| AGCG vs. GGTG | <0.001 | Yes | ** | GCAG vs. TCGC | <0.001 | Yes | * |
| GGTG vs. GCGG | <0.001 | Yes | ** | TCGC vs. GGTT | <0.001 | Yes | ** |
| GGTG vs. GTGC | <0.001 | Yes | ** | TCGC vs. GTTC | <0.001 | Yes | ** |
| GGTG vs. CGGT | <0.001 | Yes | ** | TCGC vs. TTCG | 0.758 | No | NS |
| GGTG vs. AAGC | <0.001 | Yes | ** | TCGC vs. CGCA | 0.989 | No | NS |
| AAGC vs. GCGG | <0.001 | Yes | ** | CGCA vs. GGTT | <0.001 | Yes | ** |
| AAGC vs. GTGC | <0.001 | Yes | ** | CGCA vs. GTTC | <0.001 | Yes | ** |
| AAGC vs. CGGT | 0.170 | No | NS | CGCA vs. TTCG | 0.969 | No | NS |
| CGGT vs. GCGG | <0.001 | Yes | ** | TTCG vs. GGTT | <0.001 | Yes | ** |
| CGGT vs. GTGC | <0.001 | Yes | ** | TTCG vs. GTTC | <0.001 | Yes | ** |
| GTGC vs. GCGG | <0.001 | Yes | ** | GTTC vs. GGTT | <0.001 | Yes | ** |

Note that all the comparisons not shown in FIG. 6C were highly discriminable. In coding window 1, all neighboring codon pairs except the Codon_{12/16} and Codon_{16/14} pairs were highly discriminable or discriminable. However, the Codon_{12/14} pair was discriminable. Therefore, except Codon₁₆ (ACAA), all the other five codons, Codon₁₁ through Codon₁₅ (GTTT, TTTT, TTTA, TTAC, and TACA) were discriminated from each other. In coding window 2, all neighboring codon pairs except the Codon_{25/23/24} cluster were highly discriminable or discriminable, suggesting that Codon_{21, 22, 26} (ACGT, ACCC, and CGTT) and either codon from the cluster (TACC, GTTA, or TTAC) were discriminated from each other. Coding window 3 only has the Codon_{33/36} pair indiscriminate. Therefore, the four codons, Codon_{31, 32, 34, 35} (GCGG, GTGC, GGTG, and AGCG), and either codon of the Codon_{33/36} pair (CGGT or AAGC) were discriminated from each other. Coding window 4 is similar to coding window 2, with all neighboring codon pairs except the Codon_{43/45/44} cluster being highly discriminable, showing that the three codons, Codon_{41, 42, 46} (GGTT, GTTC, and GCAG) and either codon from this cluster (TTCG, CGCA, or TCGC) were discriminated from each other. Based on the codon discrimination capability (FIG. 6C), four nanopore-discriminable codons were selected among six formed in each coding window to assemble a quartet codon panel for quaternary encoding (FIG. 6D). Coding windows 1, 2, 3, and 4 were used to assemble 6, 3, 9, and 3 such quartets, respectively. Each quartet was freely selected to represent 0, 1, 2, and 3 (from low to high blocking levels) for quaternary data encoding.

### Example 14

This example presents a method for identifying and selecting coding window sequences for frameshift encoding of multinary data.

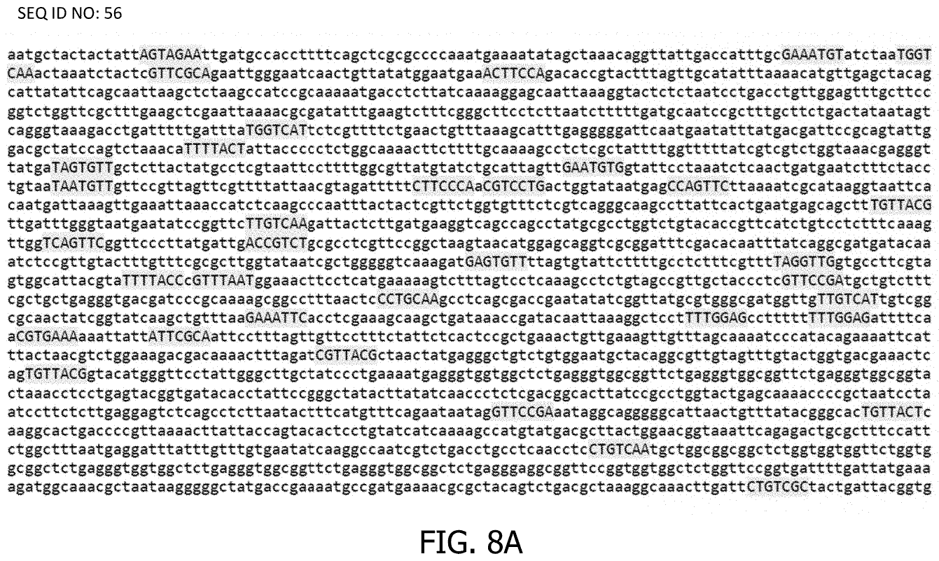

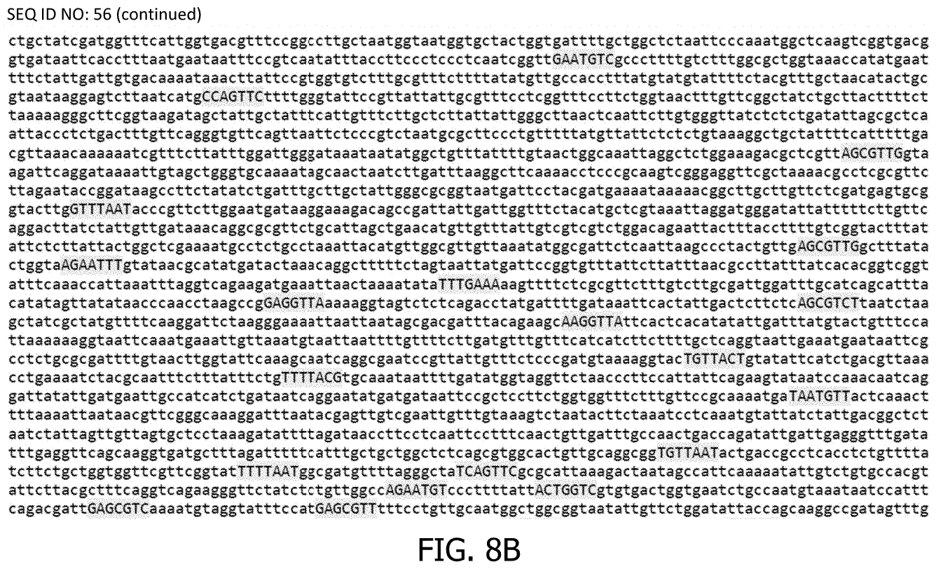

Frameshift encoding was applied in writing, re-writing, and retrieving multinary data in a long native DNA that functions as a hard drive. To use a native DNA as the universal template, it was necessary to identify Coding Window sequences according to the criteria, including capacity, step and conductance difference between two codons formed in a Coding Window (FIG. 7A). After coding windows were identified, staples were synthesized that encode multinary codons in these coding windows, followed by nanopore codon reading for data retrieval (FIG. 7B). A Python program Frame-Shift Coding Window Finder was used with the M13 DNA as a model to simulate the procedure for automatic large-scale search, selection, and design of qualified Coding Window sequences in a native DNA template. The program script is shown below,

```
import itertools
delta_x = 6
len_coden = 7
valid_seq = { }
q_values_map = { }
with open(‘Quadromer values.csv’, ‘r’) as f:
  lines = f.readlines( )
  for line in lines:
      line_array = line.split(‘,’)
      if len(line_array) == 3:
        q_values_map[line_array[0]] =
float(line_array[1])
def validate_seq(seq, delta_x):
  scores = [ ]
  sub_seqs = [ ]
  for i in range(0, len(seq) − 3):
      scores.append(q_values_map[seq[i: i+4]])
      sub_seqs.append(seq[i: i+4])
  for i1 in range(0, len(sub_seqs)):
      for i2 in range(i1 + 1, len(sub_seqs)):
        if abs(q_values_map[sub_seqs[il]] −
q_values_map[sub_seqs[i2]]) < delta_x:
          return False, [ ]
  return True, scores
# create all codens
codens=[‘’.join(x) for x in
itertools.product(‘ACGT’, repeat=len_coden)]
for seq in codens:
  is_valid, scores = validate_seq(seq.strip( ),
delta_x)
  if is_valid is True:
      valid_seq[seq] = scores
print(‘Valid sequence count:
{ }’.format(len(valid_seq)))
with open(‘output.csv’, ‘w’) as f:
  for k in valid_seq.keys( ):
      f.write(k + ‘,’ + ‘,’.join([str(s) for s in
valid_seq[k]]) + ‘\n’)
with open(‘m13.txt’, ‘r’) as file_m13:
  seq_m13 = file_m13.read( )
  print(“Length of m13:”,len(seq_m13))
  count = 0
  for vs in valid_seq.keys( ):
      count = count + seq_m13.count(vs)
      print(vs,seq_m13.count(vs))
      # seq_m13 = seq_m13.replace(vs,
‘\033[44;33m{ }\033[m’.format(vs))
      seq_m13 = seq_m13.replace(vs, vs.lower( ))
  print(“Coden number in M13:”,count)
  print(“Coded M13:”+‘\n’+ seq_m13)
  print(len_coden, “−bit coden,”, “Delta
current=”, delta_x, “,”, ‘Valid coden count:
{ }’.format(len(valid_seq)))
  with open(“m13_coded.txt”, “w”) as file_coded:
    file_coded.write(seq_m13)
```

To encode an n-nary dataset (n is 2, 4, 8 and 16 for binary, quaternary, octal and hexadecimal data), each coding window must be able to form n different (nanopore-discriminable) codons by frameshift, i.e. the Capacity is n. The resulting coding window sequences were also dependent on the number of bases for each shift, i.e. Step. Step can be set to, but not limited to 1. The search with multiple Step values resulted in more qualified candidate sequences. If the frameshift step is set to 1-nt, a coding window needs to contain at least n+3 nucleotides. Most importantly, the nanopore current difference produced between any two codons should be larger than a cut-off value ΔI_{shift}, a measure of discriminability at the instrument resolution. A large ΔI_{shift} increased codon discrimination accuracy, but also caused fewer qualified candidates.

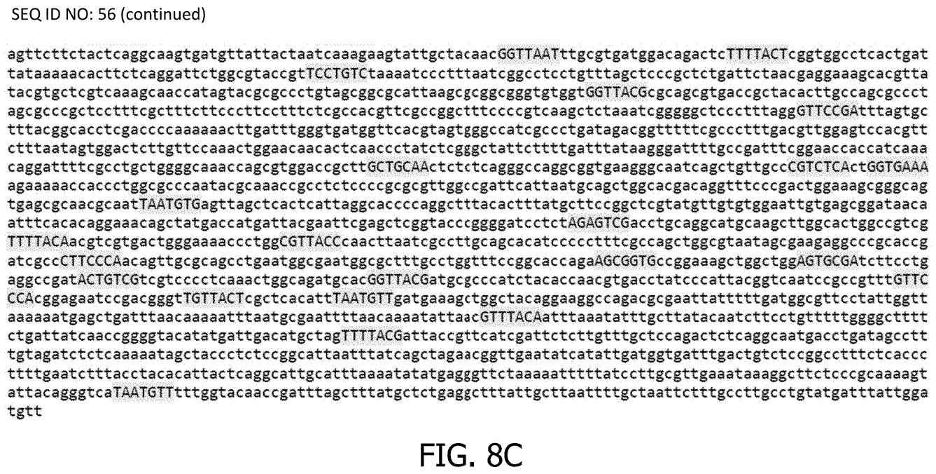

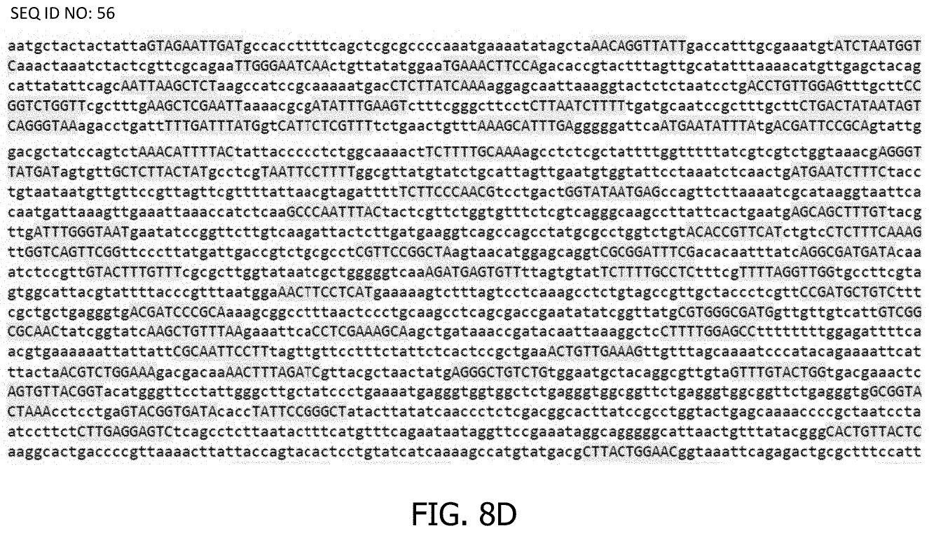

First, how to select qualified coding window sequences for encoding quaternary data was shown. The sequence of M13mp18 (7249 nucleotides) was obtained from New England Biolabs' website (SEQ ID NO: 56). The criteria for searching criteria for Coding Window sequences include: (i) Codon length=4 bases, (ii) Capacity=4 bases, (iii) Step=1-nt, and (iv) ΔI_{shift}=6 pA. All the coding window sequences satisfying these criteria were 7-nt long and formed 4 codons by frameshift that have a >ΔI_{shift}=6 pA between any two codons among four. ΔI_{shift} were calculated based on the current levels of all 256 quadromers obtained from the previous work, which used the enzyme phi29 DNAP to control stepwise ssDNA translocation and measured at 180 mV in 0.3 M KCl in both side at pH 8.0) (Laszlo et al., 2014), a different condition from that in the current work. The screening based on the above condition finally identified 78 7-nt coding window sequences and highlighted in the M13 DNA sequence (FIGS. 8A-8C, SEQ ID NO: 56). Considering the address sequences (for binding with staples) that are about 20-nt long, the M13 DNA was estimated to provide about 70 coding positions for quaternary encoding (FIGS. 8A-8C).

The program was further used to simulate the coding window screening in M13 DNA for octal encoding. The criteria for searching criteria for Coding Window sequences include: (i) Codon length=4 bases, (ii) Capacity=8, (iii) Step=1-nt, and (iv) ΔI_{shift}=1 pA. Each coding window should contain 8+3=11 nucleotides. Under these conditions, 201 coding windows were identified and highlighted in the M13 DNA sequence (FIGS. 8C and 8D, SEQ ID NO: 56). Coding windows in sequences truncated from M13 DNA for quaternary and octal encoding have been successfully tested.

FIGS. 7A and 7B. The schematics of the automatic identification of coding windows in a native DNA sequence and encoding data into the sequence (M13 genomic DNA) by these coding windows. FIG. 7A. A schematic for identifying coding windows in a native DNA sequence. FIG. 7B. A schematic for encoding data into the M13 genomic DNA by these coding windows.

FIGS. 8A-8F. M13 DNA with highlighted coding windows. FIGS. 8A-8C. M13 DNA (SEQ ID NO: 56) with 78 highlighted coding window sequences for quaternary encoding. FIGS. 8D-8F. M13 DNA (SEQ ID NO: 56) with 201 highlighted coding window sequences for octal encoding.

### Example 15

This example presents an example of multinary data encoding and decoding by NP Unzip-Seq.

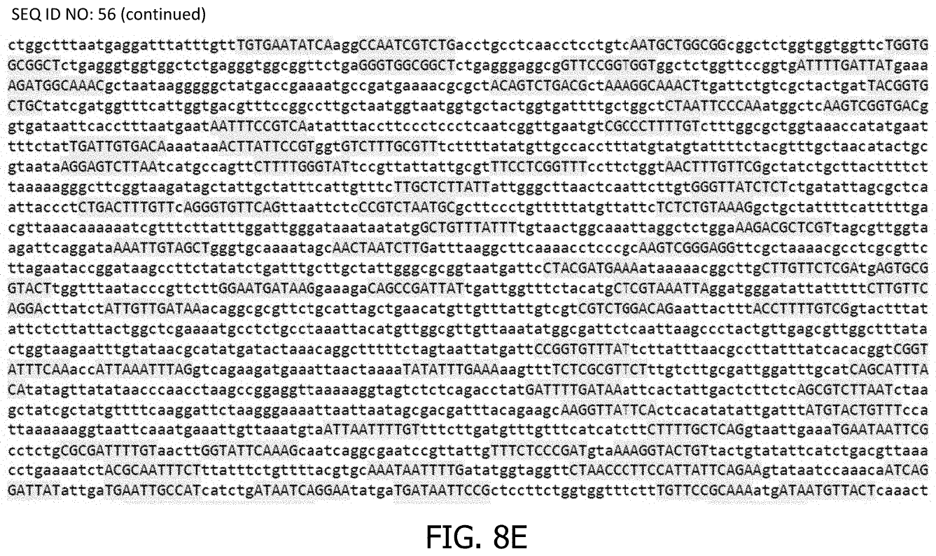

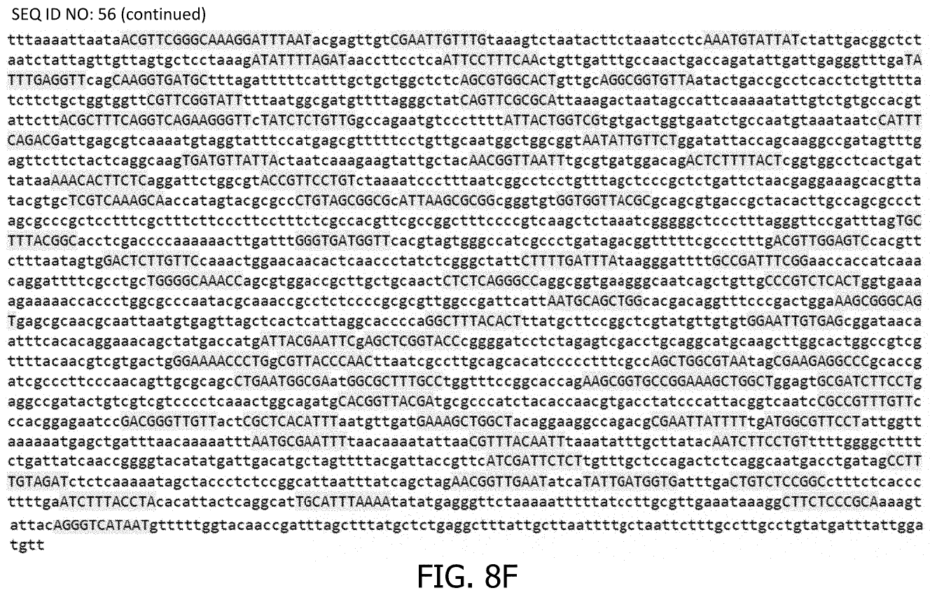

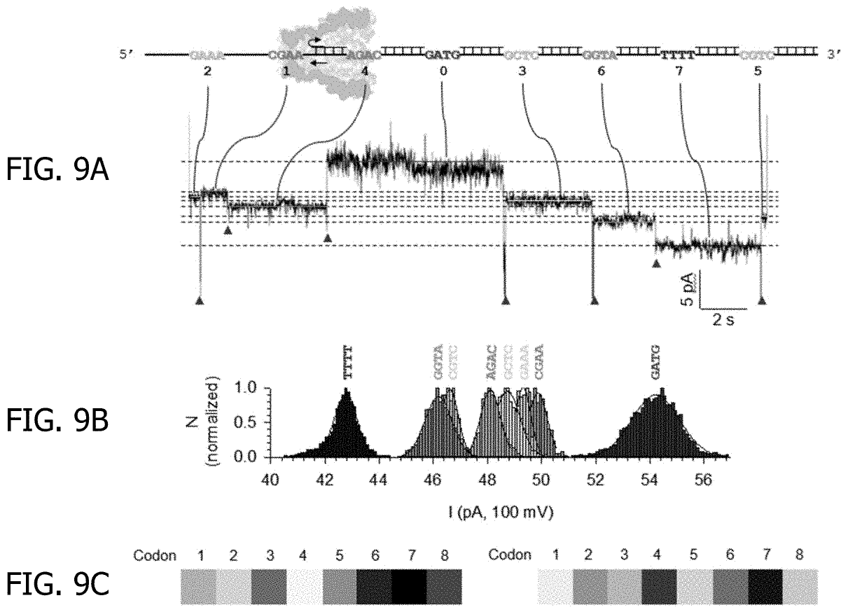

A data DNA was designed with eight different codons (D-Octal) to simulate octal encoding (FIG. 9A). These codons, including GAAA, CGAA, AGAC, GATG, GCTC, GGTA, TTTT and CGTC, were selected based on their conductance in the MspA pore, and were permutated in the template in descending order of the conductance. Each codon was linked to the same blocker segment to form the duplex blocker with the universal staple. The signature for vectorial unzipping of the codon-duplex complex (FIG. 9A) showed seven inter-codon markers (marked by triangles) that split the signature into eight sequential stages, each at a specific current level. Therefore, the observed eight stages were assigned to the 8 codons, which were sequentially read by the nanopore as the codon-associated duplex blockers were unzipped one by one. The eight codons were discriminated from each other by their current levels (FIG. 9B), from high to low, 23.86% for GATG, 22.40% for CGAA, 22.15% for GAAA, 21.89% for GCTC, 21.63% for AGAC, 20.98% for CGTC, 20.70% for GGTA, 19.27% for TTTT. This result showed that, in addition to the codon sequences, other factors such as molecular configurations in the nanopore also regulated codon signatures in the nanopore. Overall, to enable multinary data decoding, it was necessary to screen all the 256 codons under various conditions including the voltage and ion strength.

In summary, the above finding verified using NP Unzip-Seq to discriminate sequential multi-nary codons, making the selected codon panel or its sub-panels a potential multinary encoding/decoding system. For example, the total eight codons were used to represent eight states for octal encoding, while selected codons among all, such as GATG, CGAA, CGTC, and TTTT, formed a sub-panel to encode quaternary information, with broad applications such as image storage where each codon encodes a grey level or a color. As such, the nanopore functions as a multi-pixel image decoder (FIG. 9C).

FIGS. 9A-9C. Reading multinary data via sequential discrimination of multiple codons by NP-Unzip-Seq. FIG. 9A. Schematic (top) and nanopore current signature (bottom) for reading the eight codons from the D-Octal by NP Unzip-Seq. Eight codons were GAAA, CGAA, AGAC, GATG, GCTC, GGTA, TTTT, and CGTC, with a descent order of conductance. Each codon was coupled with a universal duplex blocker. The 5′ end was extended with 6C to head into the nanopore. The current levels for the eight codons are marked by horizontal lines and seven inter-codon markers separating the eight codons are marked by triangles; FIG. 9B. The current histograms of the eight codons. According to the current levels observed using NP Unzip-Seq, these codons represent the eight octonary values, GAAA (49.3 pA) for 2, CGAA (49.9 pA) for 1, AGAC (48.1 pA) for 4, GATG (54.3 pA) for 0, GCTC (48.7 pA) for 3, GGTA (46.1 pA) for 6, TTTT (42.8 pA) for 7, and CGTC (46.7 pA) for 5; FIG. 9C. Diagram showing an octonary 8-pixel grey level image (left) and grey-scale color image (right) encoded by D-Octal.

### Example 16

This example demonstrates advantages of this invention as a synthesis-free DNA data writing/reading strategies.

This method utilized a set of unmodified staples to selectively recode (or translate) the template sequence into different codon sequences for writing various binary and multinary target datasets. It offered high precision and capacity in data writing by single-base manipulation. Shifting a single base was sufficient to generate completely different codons, allowing a short coding window to generate the desired number of codons for multinary data writing. For example, a short 5-, 7-, 11-, or 19-nt (n) coding window in the template sequence could generate 2, 4, 8, or 16 (n-3) quadromeric codons by 1-nt step frameshifting, to represent all bit values in binary, quaternary, octal, or hexadecimal data. Conceptually different from other data writing approaches, Frameshift Encoding does not need an enzymatic or chemical synthesis of long DNA. This hybridization-based writing strategy only needs a universal DNA template and a staple pool, which is then used for any data writing, thus is both rapid and cost-saving. First, since this data storage method encoded data by exposing the information hiding in the universal DNA, there was no need to introduce protein tags or other labels to produce the coding signal. Secondly, it did not need to read the data by next-generation sequencing, which still is a sequencing-by-synthesis technology. Third, the capacity of the data writing was highly enhanced by frameshifting multinary codons and the small size of the staples (20˜30 nt). Fourth, it did not involve any enzyme, thus eliminating the concern about the enzyme specificity and efficiency in both the writing and reading processes. Lastly, the simple mix-then-read mode without any chemical or enzymatic reaction further significantly decreased the time and cost. Overall, Frameshift Encoding represents a new model of DNA hard drive that can use native long genome DNAs as templates for synthesis-free, label-free, rapid, low-cost, rewritable, high-density, multinary information storage.

This was used to develop native DNAs as universal templates. The single-stranded M13 DNA remains a preferred model for early-stage exploration because data was directly written by hybridization with staples. In other systems, long single-stranded templates are extracted from double-stranded DNAs by approaches such as asymmetric PCR, denatured electrophoresis, and enzymatic digestion. The most important issue is identifying coding windows in the native DNA sequence for Frameshift Encoding. To write a multinary (n=2, 4, 8, and 16) dataset by frameshifting, each coding window needs to generate n different codons, and these codons need to be nanopore-discriminable. Therefore, it is a priority to screen all the 4⁴=256 quadromeric codons in the nanopore, characterizing their signatures and evaluating their nanopore discriminability, requiring the facilitation of high throughput nanopore devices due to a large number of parallel tests (over 1,000). The outcome from such screening test would be a 256×256 discriminability chart, in which each codon-pair is ranked as discriminable or indiscriminate, useful for coding window design and search. The result would also vary with different detection methods and conditions, such as the salt concentration, pH and the voltage applied. Simulation work envisions a process for an automatic large-scale search of qualified coding windows in native DNA (FIG. 7A, FIG. 7B). For example, to encode octal data (n=8) and by setting the cut-off blocking level difference between any two codons to 1 pA, 201 11-nt octal coding windows were identified in the 7249-nt M13 DNA (FIGS. 8A-8F).

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the disclosure are achieved and other advantageous results attained.

As various changes could be made in the above methods, processes, and compositions without departing from the scope of the disclosure, it is intended that all matter contained in the above description and shown in the accompanying drawings shall be interpreted as illustrative and not in a limiting sense.

1. A method for reading stored data, the method comprising:
directing a portion of a nucleic acid chain into a nanopore, wherein the chain represents stored data and comprises codons, addresses, and blockers, wherein the codons each comprise one or more unpaired nucleotides and wherein the addresses and the blockers each comprise segments of nucleotides, wherein the blockers are each configured to be bound with a corresponding one of the addresses, and wherein the nanopore comprises a constricting region;
applying an electric potential across the nanopore to move the chain through the nanopore, wherein the chain moves through the nanopore until a first codon of the chain enters the constricting region and a first blocker of the chain and a corresponding first address of the chain bound thereto encounters the constricting region, and wherein the first blocker and its corresponding first address encountering the constricting region stops the movement of the chain;
measuring a first current in the nanopore when the first codon is in the constricting region;
dissociating the first blocker from its corresponding first address bound thereto to resume the movement of the chain through the nanopore;
repeating the measuring step and the dissociating step with additional codons to measure additional currents; and
translating the measured currents into an output signal representative of the stored data.

2. The method of claim 1 wherein the method further comprises measuring a second current after the first blocker dissociates from its corresponding first address and before a second codon enters the constricting region.

3. The method of claim 2, wherein the second current is used to demarcate the first current and a third current associated with the second codon.

4. The method of claim 1, further comprising identifying one or more repeating patterns in the measured currents to identify one or more repeating codons in the chain.

5. The method of claim 1, wherein the first current of the first codon has a characteristic current pattern associated with the sequence of the nucleotides of the first codon.

6. The method of claim 1, wherein the codons each comprise 2 or more nucleotides.

7. The method of claim 6, wherein the codons each comprise 4 or 5 nucleotides.

8. The method of claim 1, wherein the addresses each comprise about 5 to about 30 nucleotides.

9. The method of claim 1, wherein the nucleic acid is used for the labelling and/or identification of a biomarker.

10. The method of claim 1, wherein the nucleic acid chain comprises a native nucleic acid.

11. The method of claim 1, wherein dissociating the first blocker from its corresponding first address comprises performing enzyme-free vectorial unzipping.

12. The method of claim 1, wherein the electric potential applied across the nanopore is between 50 mV to 200 mV.

13. The method of claim 1, wherein the output signal is binary.

14. The method of claim 1, wherein the output signal is quaternary, octal, or hexadecimal.

15. A method for encoding data and reading the encoded data, the method comprising
providing a nucleic acid chain, wherein the chain comprises a plurality of coding windows and addresses, wherein the coding windows each comprise three or more unpaired nucleotides and the addresses each comprise three or more unpaired nucleotides;
binding the addresses to blockers, wherein the blockers each comprise an address match and an encoder, the address match complements the nucleotides of the addresses, and the encoder complements nucleotides of the coding windows adjacent to the addresses;
defining codons based on the address match and the encoder to encode data into at least a portion of the chain, wherein the codons each comprise two or more nucleotides of the coding window preceding the nucleotides bound with the blocker; and
reading the encoded data using the method of claim 1.

16. The method of claim 15, wherein the nucleotides of the coding windows have a coding window sequence and the coding window sequence is the same for all the coding windows.

17. The method of claim 15, wherein the nucleotides of the addresses have an address sequence and the chain comprises two or more address sequences.

18. The method of claim 15, wherein defining codons based on the address match and the encoder comprises shifting the coding window along the chain by a predetermined number of bits.

19. The method of claim 15, wherein the encoder comprises 0 or more nucleotides and wherein a size of the encoder determines the number of bits by which the coding window is shifted.

20. A kit for writing and reading data, the kit comprising:
a universal nucleic acid chain comprising coding windows and addresses, wherein the coding windows comprise three or more unpaired nucleotides and the addresses comprise three or more unpaired nucleotides;
blockers comprising an address match and an encoder, wherein the address match comprises nucleotides complementing the nucleotides of the addresses and the encoder comprises nucleotides complementing nucleotides of the coding windows adjacent to the addresses, and the blockers are configured such that they may bind to the addresses and thereby define codons in the coding windows such that the codons comprise two or more nucleotides of the coding window preceding the nucleotides bound with the blocker;
a microfluidic device comprising:
an inlet for receiving a flow comprising the chain, and
a plurality of nanopores comprising a constricting region configured such that applying an electric potential across the nanopore causes the chain to move through the nanopore until a first codon enters the constricting region and a first blocker encounters the constricting region and temporarily stops the movement of the chain; and
a measuring device for measuring the current through the nanopore.


SEQUENCE LISTING (57 sequences)

SEQ ID NO: 1 - DNA, 11 bp, Artificial Sequence
  Synthetic
gtttgttccca

SEQ ID NO: 2 - DNA, 29 bp, Artificial Sequence
  Synthetic
tttttttttccagcatgtacttctcgacc

SEQ ID NO: 3 - DNA, 29 bp, Artificial Sequence
  Synthetic
ttttttttcccagcatgtacttctcgacc

SEQ ID NO: 4 - DNA, 29 bp, Artificial Sequence
  Synthetic
tttttttccccagcatgtacttctcgacc

SEQ ID NO: 5 - DNA, 29 bp, Artificial Sequence
  Synthetic
ttttttcccccagcatgtacttctcgacc

SEQ ID NO: 6 - DNA, 29 bp, Artificial Sequence
  Synthetic
tttttccccccagcatgtacttctcgacc

SEQ ID NO: 7 - DNA, 29 bp, Artificial Sequence
  Synthetic
ttttcccccccagcatgtacttctcgacc

SEQ ID NO: 8 - DNA, 29 bp, Artificial Sequence
  Synthetic
tttccccccccagcatgtacttctcgacc

SEQ ID NO: 9 - DNA, 29 bp, Artificial Sequence
  Synthetic
ttcccccccccagcatgtacttctcgacc

SEQ ID NO: 10 - DNA, 29 bp, Artificial Sequence
  Synthetic
tccccccccccagcatgtacttctcgacc

SEQ ID NO: 11 - DNA, 29 bp, Artificial Sequence
  Synthetic
cccccccccccagcatgtacttctcgacc

SEQ ID NO: 12 - DNA, 20 bp, Artificial Sequence
  Synthetic
ggtcgagaagtacatgctgg

SEQ ID NO: 13 - DNA, 20 bp, Artificial Sequence
  Synthetic
ggtcgtacatgaagagctgg

SEQ ID NO: 14 - DNA, 80 bp, Artificial Sequence
  Synthetic
ccccccccccccagcatgtacttctcgacccccccccagcatgtacttctcgacccccccccagcatgtacttctcgacc

SEQ ID NO: 15 - DNA, 80 bp, Artificial Sequence
  Synthetic
ccccccccccccagcatgtacttctcgacccccccccagcatgtacttctcgacctttttccagcatgtacttctcgacc

SEQ ID NO: 16 - DNA, 80 bp, Artificial Sequence
  Synthetic
ccccccccccccagcatgtacttctcgacctttttccagcatgtacttctcgacccccccccagcatgtacttctcgacc

SEQ ID NO: 17 - DNA, 80 bp, Artificial Sequence
  Synthetic
ccccccccccccagcatgtacttctcgacctttttccagcatgtacttctcgacctttttccagcatgtacttctcgacc

SEQ ID NO: 18 - DNA, 80 bp, Artificial Sequence
  Synthetic
ccccctttttccagcatgtacttctcgacccccccccagcatgtacttctcgacccccccccagcatgtacttctcgacc

SEQ ID NO: 19 - DNA, 80 bp, Artificial Sequence
  Synthetic
ccccctttttccagcatgtacttctcgacccccccccagcatgtacttctcgacctttttccagcatgtacttctcgacc

SEQ ID NO: 20 - DNA, 80 bp, Artificial Sequence
  Synthetic
ccccctttttccagcatgtacttctcgacccttttccagcatgtacttctcgacccccccccagcatgtacttctcgacc

SEQ ID NO: 21 - DNA, 80 bp, Artificial Sequence
  Synthetic
ccccctttttccagcatgtacttctcgacctttttccagcatgtacttctcgacctttttccagcatgtacttctcgacc

SEQ ID NO: 22 - DNA, 200 bp, Artificial Sequence
  Synthetic
cgatgcctgctgctctgaccccccccctgctgctctgacctttttcctgctgctctgaccccccccctgctgctctgaccccccccctgctgctctgacctttttcctgctgctctgacctttttcctgctgctctgacctcccccctgctgctctgacctttttcctgctgctctgacccgatgcctgctgctctgacc

SEQ ID NO: 23 - DNA, 15 bp, Artificial Sequence
  Synthetic
ggtcagagcagcagg

SEQ ID NO: 24 - DNA, 90 bp, Artificial Sequence
  Synthetic
tttttcccccccagcatgtacttctcgacctttttcccccggatttcaagttctccctcctttttcccccgctcttcaaggtgcacatgg

SEQ ID NO: 25 - DNA, 25 bp, Artificial Sequence
  Synthetic
ggtcgagaagtacatgctggggggg

SEQ ID NO: 26 - DNA, 20 bp, Artificial Sequence
  Synthetic
ggagggagaacttgaaatcc

SEQ ID NO: 27 - DNA, 25 bp, Artificial Sequence
  Synthetic
ggagggagaacttgaaatccggggg

SEQ ID NO: 28 - DNA, 20 bp, Artificial Sequence
  Synthetic
ccatgtgcaccttgaagagc

SEQ ID NO: 29 - DNA, 25 bp, Artificial Sequence
  Synthetic
ccatgtgcaccttgaagagcggggg

SEQ ID NO: 30 - DNA, 32 bp, Artificial Sequence
  Synthetic
cctttttcccccggatttcaagttctccctcc

SEQ ID NO: 31 - DNA, 100 bp, Escherichia virus M13
cgttttacaacgtcgtgactgggaaaacgttacccaacttaatcgccttgcaagcggtgccggaaagctggctgggttcgcagaattgggaatcaactgt

SEQ ID NO: 32 - DNA, 25 bp, Artificial Sequence
  Synthetic
acgttttcccagtcacgacgttgta

SEQ ID NO: 33 - DNA, 21 bp, Artificial Sequence
  Synthetic
ttttcccagtcacgacgttgt

SEQ ID NO: 34 - DNA, 24 bp, Artificial Sequence
  Synthetic
aacgttttcccagtcacgacgttg

SEQ ID NO: 35 - DNA, 23 bp, Artificial Sequence
  Synthetic
aacgttttcccagtcacgacgtt

SEQ ID NO: 36 - DNA, 22 bp, Artificial Sequence
  Synthetic
aacgttttcccagtcacgacgt

SEQ ID NO: 37 - DNA, 20 bp, Artificial Sequence
  Synthetic
acgttttcccagtcacgacg

SEQ ID NO: 38 - DNA, 25 bp, Artificial Sequence
  Synthetic
gcttgcaaggcgattaagttgggta

SEQ ID NO: 39 - DNA, 24 bp, Artificial Sequence
  Synthetic
gcttgcaaggcgattaagttgggt

SEQ ID NO: 40 - DNA, 23 bp, Artificial Sequence
  Synthetic
gcttgcaaggcgattaagttggg

SEQ ID NO: 41 - DNA, 22 bp, Artificial Sequence
  Synthetic
gcttgcaaggcgattaagttgg

SEQ ID NO: 42 - DNA, 21 bp, Artificial Sequence
  Synthetic
gcttgcaaggcgattaagttg

SEQ ID NO: 43 - DNA, 20 bp, Artificial Sequence
  Synthetic
gcttgcaaggcgattaagtt

SEQ ID NO: 44 - DNA, 15 bp, Artificial Sequence
  Synthetic
ccagccagctttccg

SEQ ID NO: 45 - DNA, 16 bp, Artificial Sequence
  Synthetic
ccagccagctttccgg

SEQ ID NO: 46 - DNA, 17 bp, Artificial Sequence
  Synthetic
ccagccagctttccggc

SEQ ID NO: 47 - DNA, 18 bp, Artificial Sequence
  Synthetic
ccagccagctttccggca

SEQ ID NO: 48 - DNA, 19 bp, Artificial Sequence
  Synthetic
ccagccagctttccggcac

SEQ ID NO: 49 - DNA, 20 bp, Artificial Sequence
  Synthetic
ccagccagctttccggcacc

SEQ ID NO: 50 - DNA, 22 bp, Artificial Sequence
  Synthetic
acagttgattcccaattctgcg

SEQ ID NO: 51 - DNA, 21 bp, Artificial Sequence
  Synthetic
acagttgattcccaattctgc

SEQ ID NO: 52 - DNA, 20 bp, Artificial Sequence
  Synthetic
acagttgattcccaattctg

SEQ ID NO: 53 - DNA, 19 bp, Artificial Sequence
  Synthetic
acagttgattcccaattct

SEQ ID NO: 54 - DNA, 18 bp, Artificial Sequence
  Synthetic
acagttgattcccaattc

SEQ ID NO: 55 - DNA, 17 bp, Artificial Sequence
  Synthetic
acagttgattcccaatt

SEQ ID NO: 56 - DNA, 7249 bp, Escherichia virus M13
aatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttatatggaatgaaacttccagacaccgtactttagttgcatatttaaaacatgttgagctacagcattatattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattcttttgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagccttttttttggagattttcaacgtgaaaaaattattattcgcaattcctttagttgttcctttctattctcactccgctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcattaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgtt

SEQ ID NO: 57 - DNA, 20 bp, Artificial Sequence
  Synthetic
ccagcatgtacttctcgacc